(19) United States
(12) Patent Application Publication
(10) Pub. No.: US 2024/0105303 A1
(43) Pub. Date: Mar. 28, 2024

(54) PROCESSES FOR PREDICTING THERAPY BENEFITS

(71) Applicant: The Wistar Institute of Anatomy and Biology, Philadelphia, PA (US)

(72) Inventors: Noam Auslander, Philadelphia, PA (US); Andrew Patterson, Philadelphia, PA (US)

(21) Appl. No.: 18/469,813

(22) Filed: Sep. 19, 2023

Related U.S. Application Data
(60) Provisional application No. 63/376,179, filed on Sep. 19, 2022.

(51) Int. Cl. G16H 20/10

(52) U.S. Cl. CPC G16H 20/10

(57) ABSTRACT

Described are systems and methods of predicting a response to a medical treatment in a subject. The systems and methods include the steps of selecting a set of mutations within at least one biological process, training a set of classifiers from the set of selected mutations via a training dataset, determining the performance level of each classifier via a validation dataset, applying a subset of high-performance level classifiers from the validation dataset via a test dataset, and predicting the response to the medical treatment based on the test dataset.

### CROSS-REFERENCE TO RELATED APPLICATIONS

This application claims priority to U.S. Provisional Application No. 63/376,179, filed on Sep. 19, 2022, incorporated herein by reference in its entirety.

### STATEMENT REGARDING FEDERALLY SPONSORED RESEARCH OR DEVELOPMENT

This invention was made with government support under Grant Numbers R00CA252025 and P50CA174523, awarded by the National Institutes of Health. The government has certain rights in the invention.

### BACKGROUND OF THE INVENTION

Melanoma is a highly aggressive disease and the deadliest form of skin cancer. Deaths from melanoma account for approximately 60% of skin cancer mortality (see American Cancer Society, “Cancer Facts & Figures 2021”, 2021; and American Cancer Society, “Cancer Facts & Figures 2017”, 2017). Prognosis greatly depends on the stage at which the cancer is discovered. Whereas almost all patients diagnosed with localized melanoma survive for at least five years, less than a third of patients diagnosed with distant metastasized melanoma survive over the same period (see American Cancer Society, “Survival Rates for Melanoma Skin Cancer”, 2021). The majority of patients with metastatic melanoma do not benefit from surgery, chemotherapy and radiation alone (see Bhatia, S., et al., Oncol., 2009; and Domingues, B., et al., ImmunoTargets Ther., 2018). Targeted therapies such as BRAF and MEK inhibitors have dramatically improved prognosis of patients with metastatic melanoma that harbor specific mutations (see Jardim, D. L., et al., Cancer Cell, 2021; and Sharma, P., et al., Science, 2015). However, only a subset of the patients can benefit from these treatments, and the majority of those develop resistance over time (see Sharma, P., et al., Science, 2015; and Villanueva, J. et al. Cancer Cell, 2010). In recent years, Immune Checkpoint Inhibitor (ICI) therapy has been approved for patients with advanced disease, demonstrating durable remission in up to half of the patients (see Domingues, B., et al., ImmunoTargets Ther., 2018; Sharma, P., et al., Science, 2015; and Larkin, J. et al. N. Engl. J. Med., 2015).

The first antibody developed for clinical ICI treatment targets the cytotoxic T-lymphocyte antigen 4 (CTLA-4). CTLA-4 is a T-cell surface protein which binds to B7-1 and B7-2 expressed by antigen-presenting cells (APC) (see Gide, T. N., et al., Clin. Cancer Res, 2018), resulting in suppression of immune response by the T-cells. Ipilimumab, a human monoclonal antibody targeting CTLA-4, was the first ICI agent to demonstrate increased progression free survival (PFS) and overall survival (OS) compared to more traditional cancer treatment methods (see Gide, T. N., et al., Clin. Cancer Res, 2018; Hodi, F. S. et al., N. Engl. J. Med., 2010; Robert, C. et al. N. Engl. J. Med., 2011). Subsequently, clinical targeting of the programmed cell death receptor 1 (PD-1), which binds to its ligand receptor PD-L1 to elicit tumor immune escape, has markedly improved the treatment of melanoma and demonstrated durable responses in other types of cancer. Several potential new ICI antibodies are currently being explored, such as those targeting the regulatory surface glycoprotein TIM-3 (see Friedlaender, A., et al., ESMO Open, 2019). While 40-60% of patients with advanced melanoma experience benefit from ICI, a substantial fraction of patients do not benefit from this treatment, which can incur severe autoimmune adverse events (see Hodi, F. S. et al., N. Engl. J. Med., 2010; Robert, C. et al. N. Engl. J. Med., 2011; Schadendorf, D. et al., J. Clin. Oncol. Off. J. Am. Soc. Clin. Oncol., 2015; Wolchok, J. D. et al., N. Engl. J. Med., 2013). Therefore, it is critical to uncover tumor characteristics that predict response to ICI.

Numerous biomarkers have been proposed for prediction of ICI response, but most have not been validated for clinical use. Gene expression biomarkers include PDL-1 (see Gibney, G. T., et al., Lancet Oncol., 2016), CD38 (see Chen, L. et al., Cancer Discov., 2018), TIM3 (see Holderried, T. A. W. et al., Clin. Epigenetics, 2019) and CXCL9 (see House, I. G. et al., Clin. Cancer Res. Off. J. Am. Assoc. Cancer Res., 2020) expression, cytolytic activity (see Rooney, M. S., et al., Cell, 2015), as well as machine learning-derived signatures such as IPRES (see Hugo, W. et al., Cell, 2016), TIDE (see Jiang, P. et al., Nat. Med., 2018), IMPRES (see Auslander, N. et al., Nat. Med., 2018), Immonophenoscores (see Charoentong, P. et al., Cell Rep., 2017), and others (see Pérez-Guijarro, E. et al., Nat. Med., 2020; and Du, K. et al., Nat. Commun., 2021). However, recent meta-analysis evaluated the reproducibility of ICI biomarkers and found that only a subset of these maintained any predictive performance (see Litchfield, K. et al., Cell, 2021). To date, gene expression signatures predicting ICI response have not been incorporated into clinical use, likely due to limited reproducibility and lack of benchmarking standards, among other factors (see Byron, S. A., et al., Nat. Rev. Genet., 2016). Genomic biomarkers of ICI benefit have met more success in terms of clinical use. In 2017, FDA approved the first biomarker for anti-PD1 efficacy based on high levels of microsatellite instability (MSI-H) (see FDA, 2019). However, MSI-H is only found in a subset of gastrointestinal and endometrial tumors. In 2020, the high tumor mutation burden (TMB-H), quantifying the number of mutations in a tumor, has been approved by the FDA as a marker for anti-PD1 efficacy (see FDA, 2020). While TMB-H has been associated with ICI benefit across different cancer types, there are several challenges for its utility. For example, TMB is tumor type specific; moreover, TMB-H status does not preclude tumor progression and low TMB does not preclude response (see Jardim, D. L., et al., Cancer Cell, 2021; Xuan, J., et al., Cancer Lett., 2013). In addition, the mechanism underlying the clinical utility of the TMB is unclear. Therefore, there is a need for additional genomic ICI response biomarkers with improved predictive performance that are more biologically interpretable.

Thus there is a need in the art for improved systems and methods of predicting a response to a medical treatment in a subject. The present invention satisfies this need.

### SUMMARY OF THE INVENTION

In one aspect, a method of predicting a response to a medical treatment in a subject comprises the steps of selecting a set of biological processes, selecting a training dataset and a validation dataset, each dataset comprising a set of genome data and clinical outcomes, grouping a set of mutations into groups each corresponding to a biological process of the set of biological processes, generating a set of classifiers, each comprising a combination of mutations, to predict a clinical outcome from one of the groups of mutations, training the set of classifiers on the training dataset, calculating, with the validation dataset, a performance level of each classifier in the set of classifiers, calculating, on a test dataset comprising genome data of a subject, a predicted clinical outcome from a medical treatment on a subject based on a subset of the set of classifiers having a high performance level on the validation dataset, and treating the subject based on the predicted response to the medical treatment.

In one embodiment, the step of generating the set of classifiers comprises a Greedy forward feature selection algorithm. In one embodiment, the step of generating the set of classifiers comprises a randomized forward feature selection algorithm. In one embodiment, the step of generating the set of classifiers comprises a genetic algorithm. In one embodiment, the step of generating the set of classifiers comprises a random forest algorithm. In one embodiment, the step of generating the set of classifiers comprise a gradient boosted tree. In one embodiment, at least one classifier of the set of classifiers comprises a Forward Neural Network model. In one embodiment, at least one classifier of the set of classifiers comprises a Long Short-Term Memory Recurrent Neural Network model.

In one aspect, a system for predicting a response to a medical treatment in a subject comprises a non-transitory computer-readable medium with instructions stored thereon, which when executed by a processor perform steps comprising selecting a set of biological processes from a database of biological processes, storing a training dataset and a validation dataset on the non-transitory computer-readable medium, each dataset comprising a set of genome data and clinical outcomes, grouping a set of mutations into groups each corresponding to a biological process of the set of biological processes, generating a set of classifiers, each comprising a combination of mutations, to predict a clinical outcome from one of the groups of mutations, training the set of classifiers on the training dataset, calculating, with the validation dataset, a performance level of each classifier in the set of classifiers, calculating, on a test dataset comprising genome data of a subject, a predicted clinical outcome from a medical treatment on a subject based on a subset of the set of classifiers having a high performance level on the validation dataset, and treating the subject based on the predicted response to the medical treatment.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is an exemplary computing device.

FIG. 2 is a flowchart of an exemplary set of steps performed according to one aspect of the systems and methods disclosed herein.

FIG. 3A through FIG. 3E depict representative subsets of mutated genes within biological processes for the prediction of melanoma ICI response. FIG. 3A depicts a representative schematic of the training, validation, and testing process utilized. Mutations within each biological process were considered and used to train a classifier on the training datasets, then the performance of each classifier was evaluated through the validation dataset. Only a small subset of biological process-based classifiers with high performance on the validation datasets were further considered and applied to the test dataset. FIG. 3B and FIG. 3C depict a representative bar plot comparing the performances of different feature selection methods for the training dataset (middle panel) and the validation dataset (lower panel). The upper panel shows the genes that are most frequently selected within these processes. The dashed lines indicate random performance (thick line) and the performance of the tumor mutation burden (thin line). FIG. 3D depicts a representative boxplot comparing the validation performance between the processes that performed best and worst on the training dataset (top and bottom 5%), using the three feature-selection methods. The p-values are for one-sided rank-sum tests. Boxes show the quartiles (0.25 and 0.75) of the data, center lines show the medians, and whiskers show the rest of the distribution, excluding outliers. FIG. 3E depicts a representative correlation heatmap showing the Spearman's correlation coefficients when correlating performances (AUC) between the training and validation datasets using the three feature-selection methods.

FIG. 4A, FIG. 4B, FIG. 4C, and FIG. 4D depict representative enhancement of prediction performance of melanoma ICI response based on mutations with biological processes by application of nonlinear classifiers. FIG. 4A depicts a representative bar plot showing the Random Forest (RF) validation performances for top process-based mutation classifiers. The dashed lines indicate random performance (thick line) and the performance of the tumor mutation burden (thin line). FIG. 4B depicts representative ROC curves demonstrating the RF validation performance when using mutations within leukocyte proliferation regulation process (graph 401) and within the T-cell proliferation process (graph 402). FIG. 4C depicts a representative heatmap of the genes selected by different trees in the RF model that is trained using mutations in the leukocyte proliferation regulation process. FIG. 4D depicts representative ROC curves demonstrating the validation performances of two neural network architectures (Long Short-Term Memory Recurrent Neural Network (LSTM) and Forward Neural Network (FNN)) when trained on mutations within the hormone-mediated signaling process (graph 411), leukocyte proliferation regulation process (graph 412), and the T-cell proliferation regulation process (graph 413).

FIG. 5A and FIG. 5B depict representative prediction performance of melanoma ICI response in a model trained with a Gradient Boosting (GB) tree. FIG. 5A depicts a representative bar plot showing the GB validation performances for top pathway-based mutation classifiers. The dashed lines indicate random performance (thick line) and the performance of the tumor mutation burden (thin line). FIG. 5B depicts representative ROC curves demonstrating the GB validation performance when using mutations within the leukocyte proliferation regulation process (upper panel) and within the T-cell proliferation process (lower panel).

FIG. 6A, FIG. 6B, FIG. 6C, FIG. 6D, and FIG. 6E depict representative evaluation of the Random Forest (RF) processes classifiers. FIG. 6A depicts a representative ROC curve showing the performance of the model for the validation and Hugo test datasets when trained using mutations within the leukocyte proliferation regulation process. FIG. 6B depicts a representative ROC curve showing the performance of the model for the validation and Hugo test datasets when trained using mutations within the T-cell proliferation regulation process. FIG. 6C depicts a representative ROC curve showing the performance of the model for the validation and Hugo test datasets when trained using mutations within the hormone-mediated signaling process. FIG. 6D depicts a representative ROC curve showing the performance of the model for the validation and Hugo test datasets when trained using mutations within the ATPase binding process. FIG. 6E depicts a representative robustness analysis evaluating the validation performances of the RF process models when retrained with different random seeds. The dashed lines indicate random performance (thick line) and the performance of the tumor mutation burden (thin line).

FIG. 7A, FIG. 7B, FIG. 7C, and FIG. 7D depict representative performance of the Random Forest (RF) models and evaluation of validation performances of Gradient Boosting (GB), Long Short-Term Memory Recurrent Neural Network (LSTM), and Forward Neural Network (FNN). FIG. 7A depicts representative ROC curves showing the performances of the RF models for the training dataset. FIG. 7B, FIG. 7C, and FIG. 7D depict representative robustness analyses evaluating the validation performances of GB, LSTM, and FNN pathway models when retrained with different random seeds. The dashed lines indicate the random performance (thick line) and the performance of the tumor mutation burden (thin line).

FIG. 8A and FIG. 8B depict representative scatter plots correlating the prediction scores of the top four Random Forest (RF) classifiers that were selected in training (y-axes) to the TMB (x-axes) in the training (Liu), validation (Riaz) and test (Hugo) datasets. The Spearman's correlation coefficients are provided.

FIG. 9A, FIG. 9B, and FIG. 9C, depict a representative characterization of Random Forest (RF) classifiers and their performances. FIG. 9A depicts a representative comparison of the performances of the top four RF classifiers that were selected in training to that of the tumor mutation burden (TMB) through bootstrapping for the validation dataset. FIG. 9B depicts a representative comparison of the performances of the top four RF classifiers that were selected in training to that of the TMB through bootstrapping for the test dataset. For both 9A and 9C, p-values are calculated empirically through bootstrapping. Boxes show the quartiles (0.25 and 0.75) of the data, center lines show the medians, and whiskers show the rest of the distribution, excluding outliers. FIG. 9C depicts representative scatter plots depicting the correlation of the model AUC with the number of genes in a biological process in the training (left panel) and validation (right panel) datasets. The Spearman's correlation coefficients are provided. As evident, while correlation is substantial in the training dataset, it is weak in the validation dataset.

FIG. 10A, FIG. 10B, and FIG. 10C depict representative quantification of patient responses correctly predicted by the leukocyte proliferation regulation process Random Forrest (RF) predictor and the tumor mutation burden (TMB). FIG. 10A depicts representative quantification of true positive patients predicted in the Liu training dataset. FIG. 10B depicts representative quantification of true positive patients predicted in the Riaz validation dataset. FIG. 10C depicts representative quantification of true positive patients predicted in the Hugo test dataset.

FIG. 11A, FIG. 11B, FIG. 11C, and FIG. 11D depict representative anti-PD1 overall survival based on mutations in leukocyte proliferation and differentiation processes. FIG. 11A depicts representative Kaplan Meier survival curves comparing between anti-PD1 treated melanoma patients with high vs low prediction scores of the RF model, when trained using mutations within the leukocyte proliferation regulation process. FIG. 11B depicts representative Kaplan Meier survival curves comparing between anti-PD1 treated melanoma patients with high vs low number of selected mutations in the leukocyte differentiation process, using the genetic algorithm feature selection. FIG. 11C depicts representative coefficients of the proportional hazards RF predictor using mutations within the leukocyte proliferation regulation process, for different types of cancer. FIG. 11D depicts representative Kaplan Meier survival curves comparing between anti-PD1 treated patients with high vs low prediction scores of the RF model, when trained using mutations within the leukocyte proliferation regulation process, using an integrated data of bladder, colon, and renal cancer. For FIG. 11A, FIG. 11B, and FIG. 11D the Cox proportional hazards as well as the log-rank p-values are indicated.

FIG. 12A and FIG. 12B depict representative anti-PD1 overall survival based on mutations in hormone-mediated signaling and T-cell proliferation regulation processes. FIG. 12A depicts a representative Kaplan Meier survival curve comparing between anti-PD1 treated melanoma patients with high vs low prediction scores of the RF model when trained using the hormone-mediated signaling process. FIG. 12B depicts a representative Kaplan Meier survival curve comparing between anti-PD1 treated melanoma patients with high vs low prediction scores of the RF model when trained using the T-cell proliferation regulation process.

FIG. 13 depicts a representative ROC curve demonstrating the performance of the leukocyte proliferation regulation RF classifier when applied to the Van Allen et al. melanoma anti-CTLA4 dataset.

FIG. 14A, FIG. 14B, and FIG. 14C, depict representative overall survival in TCGA predicted by biological process-based Random Forest (RF) classifiers. FIG. 14A depicts a representative heatmap showing the hazard ratios (log-transformed) obtained through overall survival prediction using the classification scores of the selected process-based random forest classifiers. FIG. 14B depicts representative Kaplan Meier survival curves comparing between TCGA samples from four cancer types, with high vs low prediction scores of the RF model, when trained using mutations within the leukocyte proliferation regulation process. FIG. 14C depicts representative Kaplan Meier survival curves comparing between TCGA samples from four cancer types, with high vs low prediction scores of the RF model, when trained using mutations within the T cell proliferation regulation process. For FIGS. 14B and 14C the Cox proportional hazards as well as the log-rank p-values are indicated.

FIG. 15A, FIG. 15B, and FIG. 15C depict representative boxplots comparing CIBERSORT results between CTNNB1 wild-type (WT) and mutated (MUT) tumors. FIG. 15A depicts a representative boxplot comparing inferred CD8 T cells. FIG. 15B depicts a representative boxplot comparing follicular helper T cells. FIG. 15C depicts a representative boxplot comparing T regs. P-values are for one-sided rank-sum tests. Boxes show the quartiles (0.25 and 0.75) of the data, center lines show the medians, and whiskers show the rest of the distribution excluding outliers.

FIG. 16 depicts a representative correlation heatmap plot showing the Spearman's correlation coefficient correlating the leukocyte proliferation RF classifier scores with mutation signatures (y-axis) in different cancer types (x-axis).

FIG. 17A and FIG. 17B depict representative scatter plots correlating the B cell burden (BCB) and T cell burden (TCB) scores (x-axes) with the leukocyte proliferation regulation classifier scores (y-axes) for responder (blue) and non-responder (red) patients. Two-sided Spearman's Rank Correlation test coefficients and p-values are provided.

FIG. 18A and FIG. 18B depict vital statistics of subjects in the cohorts used. FIG. 18A depicts boxplots comparing the age distribution between the Memorial Sloan Kettering (MSK) cohort and the Van Allen anti-CTLA4 cohort, for which age data is available. Boxes show the quartiles (0.25 and 0.75) of the data, center lines show the medians, and whiskers show the rest of the distribution excluding outliers. FIG. 18B depicts bar plot showing the number of males vs. females in samples in the Hugo, Van Allen, MSK, and Liu cohorts where sex is available.

### DETAILED DESCRIPTION

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab and F(ab)2, as well as single chain antibodies (scFv), heavy chain antibodies, such as camelid antibodies, synthetic antibodies, chimeric antibodies, and a humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

“Cancer,” as used herein, refers to the abnormal growth or division of cells. Generally, the growth and/or life span of a cancer cell exceeds, and is not coordinated with, that of the normal cells and tissues around it. Cancers may be benign, pre-malignant or malignant. Cancer occurs in a variety of cells and tissues, including the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, meningioma, etc.), breast, genital system, (e.g., uterus, ovary, prostate, testis, etc.), urinary system (e.g., bladder, kidney, ureter, etc.), eye, nervous system (e.g., brain, etc.), endocrine system (e.g., thyroid, etc.), and hematopoietic system (e.g., lymphoma, myeloma, leukemia, acute lymphocytic leukemia, chronic lymphocytic leukemia, acute myeloid leukemia, chronic myeloid leukemia, etc.).

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered.

“Consensus” or “Consensus Sequence” as used herein may mean a synthetic nucleic acid sequence, or corresponding polypeptide sequence, constructed based on analysis of an alignment of multiple subtypes of a particular antigen. The sequence may be used to induce broad immunity against multiple subtypes, serotypes, or strains of a particular antigen. Synthetic antigens, such as fusion proteins, may be manipulated to generate consensus sequences (or consensus antigens).

The term “inhibit,” as used herein, means to suppress or block an activity or function by at least about ten percent relative to a control value. In some instances, the activity is suppressed or blocked by 50%, 75%, 90%, or 95% compared to a control value. Inhibitors are compounds that, e.g., bind to, partially or totally block stimulation, decrease, prevent, delay activation, inactivate, desensitize, or down regulate a protein, a gene, and mRNA stability, expression, function and activity, e.g., antagonists.

A “mutation,” “mutant,” or “variant,” as used herein, refers to a change in nucleic acid or polypeptide sequence relative to a reference sequence (which may be a naturally-occurring normal or the “wild-type” sequence), and includes translocations, deletions, insertions, and substitutions/point mutations. A “mutant” or “variant” as used herein, refers to either a nucleic acid or protein comprising a mutation.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Sample” or “biological sample” as used herein means a biological material isolated from a subject. The biological sample may contain any biological material suitable for detecting a mRNA, polypeptide or other marker of a physiologic or pathologic process in a subject, and may comprise fluid, tissue, cellular and/or non-cellular material obtained from the individual.

“Subject” as used herein can mean a mammal that is capable of being administered the immunogenic compositions described herein. The mammal can be, for example, a human, chimpanzee, dog, cat, horse, cow, mouse, or rat.

A “therapeutic” treatment is a treatment administered to a subject who exhibits signs of pathology, for the purpose of diminishing or eliminating those signs.

As used herein, the terms “therapy” or “therapeutic regimen” refer to those activities taken to alleviate or alter a disorder or disease state, e.g., a course of treatment intended to reduce or eliminate at least one sign or symptom of a disease or disorder using pharmacological, surgical, dietary and/or other techniques. A therapeutic regimen may include a prescribed dosage of one or more drugs or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease or disorder conditions, etc.

“Treatment” or “treating,” as used herein can mean protecting of a subject from a disease through means of preventing, suppressing, repressing, or completely eliminating the disease. In one embodiment, preventing the disease involves administering an immunogenic composition of the present invention to a subject prior to onset of the disease. In one embodiment, preventing the disease involves administering an immunogenic composition of the present invention to a subject following a treatment so as to prevent reoccurrence or further progression of the disease. Suppressing the disease involves administering an immunogenic composition of the present invention to a subject after induction of the disease but before its clinical appearance. Repressing the disease involves administering an immunogenic composition of the present invention to a subject after clinical appearance of the disease.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

Variant can further be defined as a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleotide sequence that is substantially identical over the full length of the full gene sequence or a fragment thereof. The nucleotide sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The present invention includes computing and software systems capable and configured to generate predictive response outcomes to medical treatments. In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

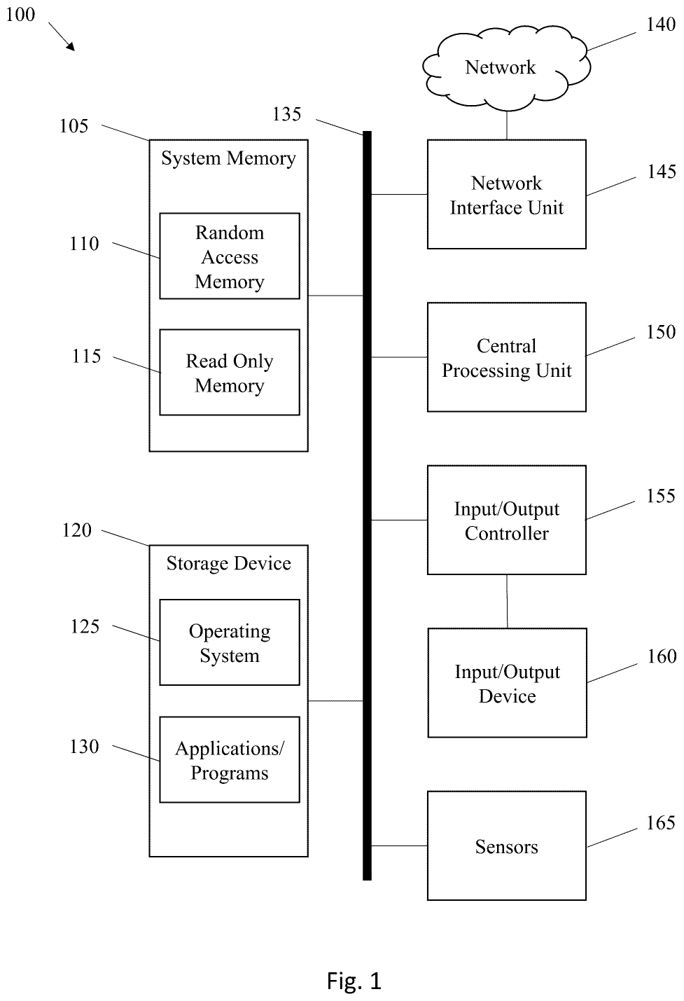

FIG. 1 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 1 depicts an illustrative computer architecture for a computer 100 for practicing the various embodiments of the invention. The computer architecture shown in FIG. 1 illustrates a conventional personal computer, including a central processing unit 150 (“CPU”), a system memory 105, including a random access memory 110 (“RAM”) and a read-only memory (“ROM”) 115, and a system bus 135 that couples the system memory 105 to the CPU 150. A basic input/output system containing the basic routines that help to transfer information between elements within the computer, such as during startup, is stored in the ROM 115. The computer 100 further includes a storage device 120 for storing an operating system 125, application/program 130, and data.

The storage device 120 is connected to the CPU 150 through a storage controller (not shown) connected to the bus 135. The storage device 120 and its associated computer-readable media provide non-volatile storage for the computer 100. Although the description of computer-readable media contained herein refers to a storage device, such as a hard disk or CD-ROM drive, it should be appreciated by those skilled in the art that computer-readable media can be any available media that can be accessed by the computer 100.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 100 may operate in a networked environment using logical connections to remote computers through a network 140, such as TCP/IP network such as the Internet or an intranet. The computer 100 may connect to the network 140 through a network interface unit 145 connected to the bus 135. It should be appreciated that the network interface unit 145 may also be utilized to connect to other types of networks and remote computer systems.

The computer 100 may also include an input/output controller 155 for receiving and processing input from a number of input/output devices 160, including a keyboard, a mouse, a touchscreen, a camera, a microphone, a controller, a joystick, or other type of input device. Similarly, the input/output controller 155 may provide output to a display screen, a printer, a speaker, or other type of output device. The computer 100 can connect to the input/output device 160 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 120 and/or RAM 110 of the computer 100, including an operating system 125 suitable for controlling the operation of a networked computer. The storage device 120 and RAM 110 may also store one or more applications/programs 130. In particular, the storage device 120 and RAM 110 may store an application/program 130 for providing a variety of functionalities to a user. For instance, the application/program 130 may comprise many types of programs such as a word processing application, a spreadsheet application, a desktop publishing application, a database application, a gaming application, internet browsing application, electronic mail application, messaging application, and the like. According to an embodiment of the present invention, the application/program 130 comprises a multiple functionality software application for providing word processing functionality, slide presentation functionality, spreadsheet functionality, database functionality and the like.

The computer 100 in some embodiments can include a variety of sensors 165 for monitoring the environment surrounding and the environment internal to the computer 100. These sensors 165 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

Application of the aforementioned systems to create and generate predictive response outcomes to medical treatments. While the system and methods described herein demonstrate significant and unexpected results as applied to immune checkpoint inhibitor (ICI) therapy for treatment of melanoma, it should be appreciated that the present invention may equally apply to other treatments and medical conditions where genetic mutation profiles and data are available.

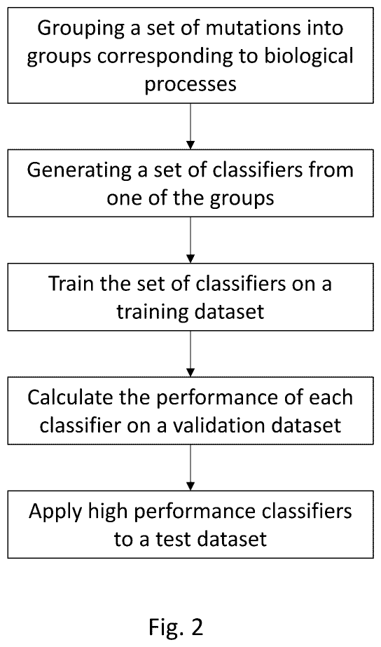

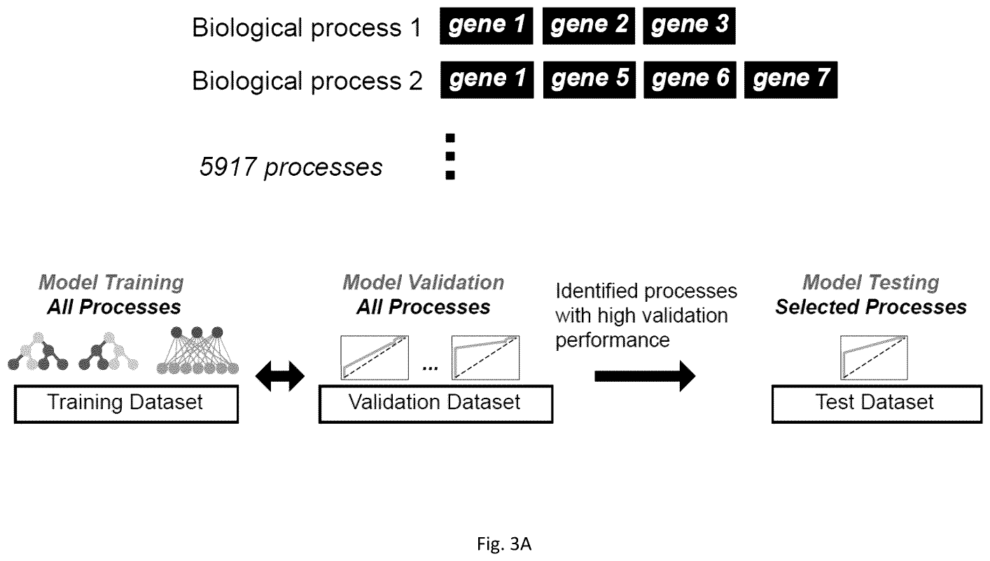

For example, as shown in FIG. 2, the systems and methods of the present invention may include and/or perform the following steps. A set of mutations is arranged in groups corresponding to at least one biological process. Next, a set of classifiers is generated from one of the groups. Next, the set of classifiers are trained from the selected set of mutations via a training dataset having known results, where predicted outcomes are compared to known results with each iteration and classifiers are refined to produce results closest to the known results of the training dataset. Next, the performance (e.g. high/low) of each classifier is calculated against a validation dataset, where the classifier is tested against other data for which the results are known, but which was not included in the training dataset. Lastly, a subset of classifiers showing high performance in the validation dataset is applied to a test dataset, for which in some embodiments the result is not known.

Optionally, subsequent treatment of a subject can be made when the predicted response to the treatment, calculated for example via one or more classifiers trained against the training dataset, is positive or otherwise favorable for the subject.

The systems and methods disclosed herein may in various embodiments use one or more different feature selection methods, alone or in combination, and including but not limited to a genetic algorithm, a forward greedy algorithm, a forward randomized algorithm. In some embodiments, prediction performance may be enhanced by applying one or more non-linear classifiers, including but not limited to a random forest and/or a gradient boosted tree. Various embodiments disclosed herein may include the training of one or more neural networking models, for example a LSTM or an FNN.

Recent studies have examined the mechanistic link between anti-PD1 response or resistance and mutated biological processes such as interferon signaling, MHC presentation and beta catenin (see Galluzzi, L., et al., Trends Cell Biol., 2019; Paschen, A., et al., Annu. Rev. Cancer Biol., 2022), prompting a need for process-level ICI response biomarkers. Here, tumor mutation data is used in the context of biological processes to predict patient response to anti-PD1 treatment.

First, the question of whether the mutation burden in genes that belong to different biological processes correlate with anti-PD1 benefit was investigated. Feature selection methods were then applied to distinct processes to identify subsets of genes in which the mutational count predicts anti-PD1 response. This revealed sets of mutated genes in several biological processes with a comparable predictive ability of anti-PD1 response to the TMB. Employing non-linear classification methods further enhanced the predictive performance of classifiers based on mutated genes in specific biological processes. The advantage of these methods is that they can capture intricate relations between the mutated genes in a process and anti-PD1 responses, simultaneously weighing mutations that contribute to either response or resistance. Evaluating decision-tree algorithms and neural network architectures, random forest maintains the most robust performance across different datasets, accurately predicting response and overall survival in independent datasets spanning over 500 melanoma patients in total. In particular, mutations in genes belonging to the leukocyte proliferation and T-cell regulation processes demonstrate consistently high predictive performances. This study provides a potential way forward for understanding ICI treatment responses and constructing biologically interpretable predictors of treatment benefit based on mutation data.

### Analysis of Mutation Sums in Biological Processes

To evaluate whether mutated genes within biological processes can predict ICI treatment responses in metastatic melanoma, training and validation mutation and clinical datasets were obtained from metastatic melanoma patients treated with anti-PD1. For all experiments, models were trained on the same designated training dataset, and evaluated using the same designated validation dataset (See methods). Throughout this work, Gene Ontology (GO) (see The Gene Ontology Consortium., Nucleic Acids Res., 2015; The Gene Ontology Consortium., Nucleic Acids Res., 2019) was used to aggregate genes into biological processes. It was first investigated whether the mutation load in genes belonging to distinct biological processes can accurately predict ICI responses. For each GO biological process, the number of mutations in that process per sample were counted in the training datasets and these values used to predict anti-PD1 responses. These analyses revealed that the total mutation counts in distinct biological processes were only mildly predictive of response.

### Linear Selection of Mutation Subsets in Biological Processes

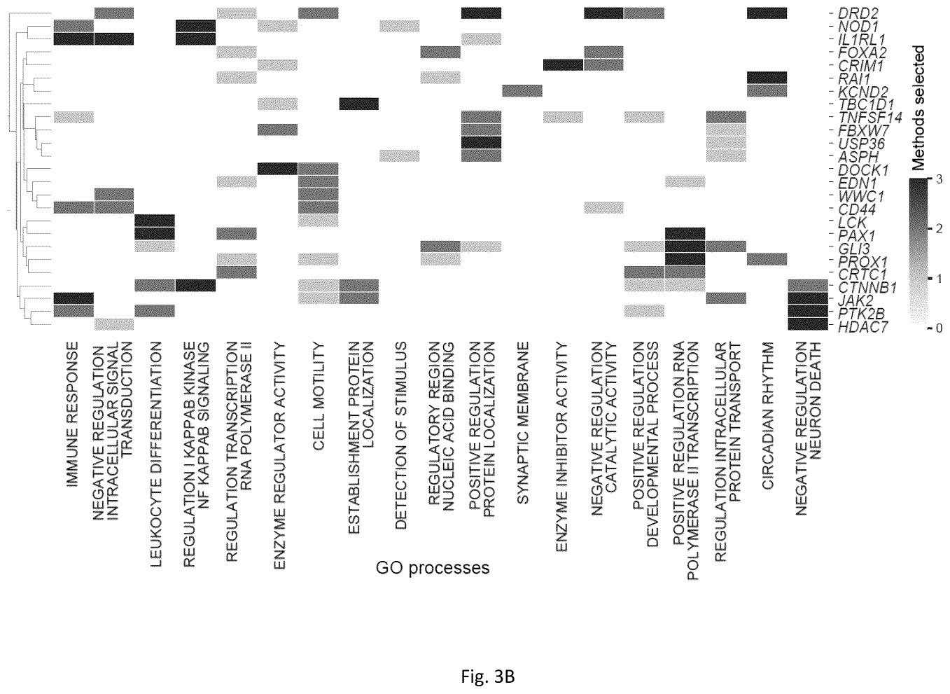

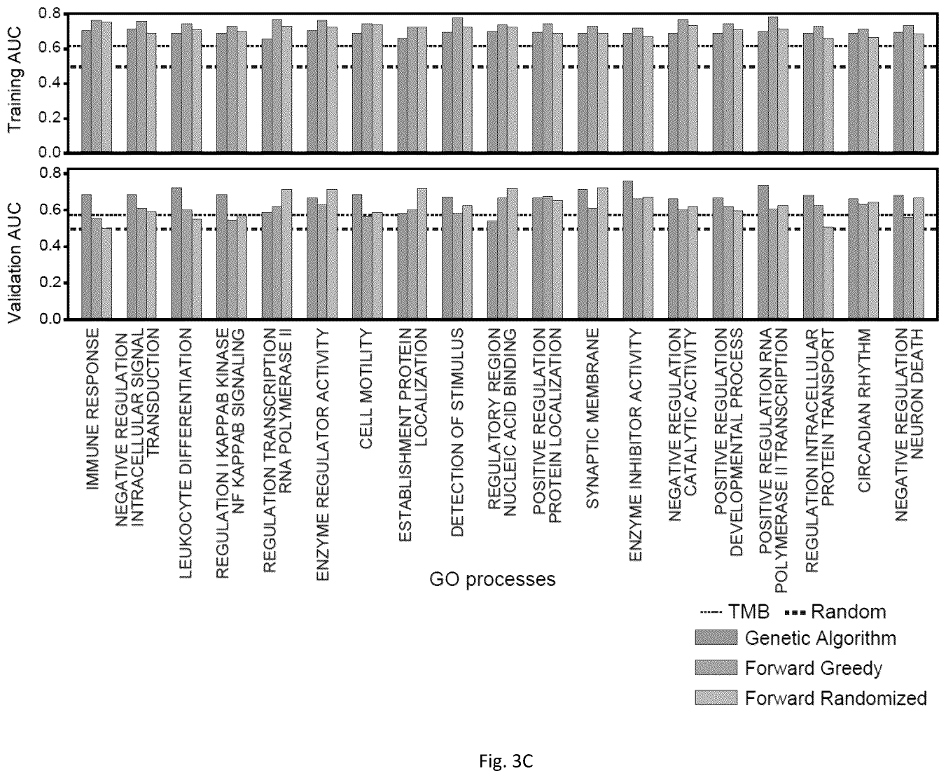

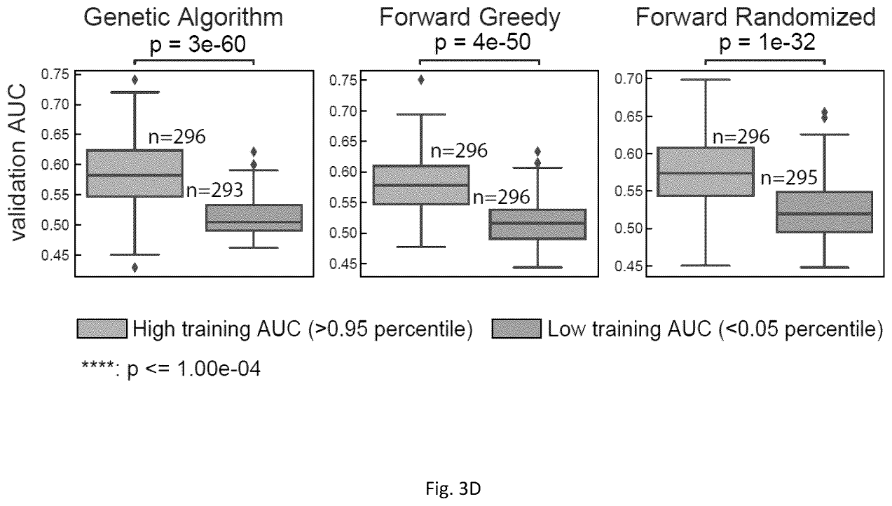

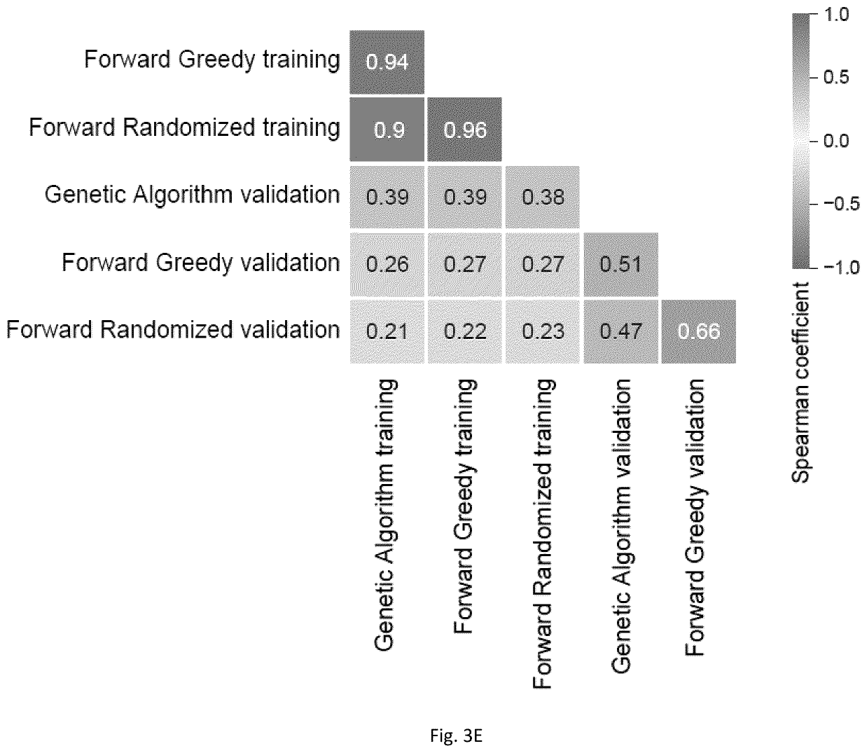

To identify subsets of genes within distinct biological processes in which the mutation count best predicts ICI response, feature selection methods were applied to mutations in each biological process. The sum of mutations in selected subsets of genes within distinct biological processes were used to predict melanoma ICI responders vs. non-responders. The area under the receiver operating characteristic curve (ROC AUC) was used to evaluate the predictive capacity of mutations in subsets of genes belonging to each biological process. A training dataset was used to build a classification model, and a validation dataset to select biological process-based models with high ICI predictive performance. Both the training and validation datasets are therefore considered part of the training process, in which all biological processes are examined. The subset of biological process-based classifiers that yield substantially better ICI predictive performance compared to the TMB on both the training and validation datasets were later evaluated on independent test datasets, as illustrated in FIG. 3A (See below for information about each dataset).

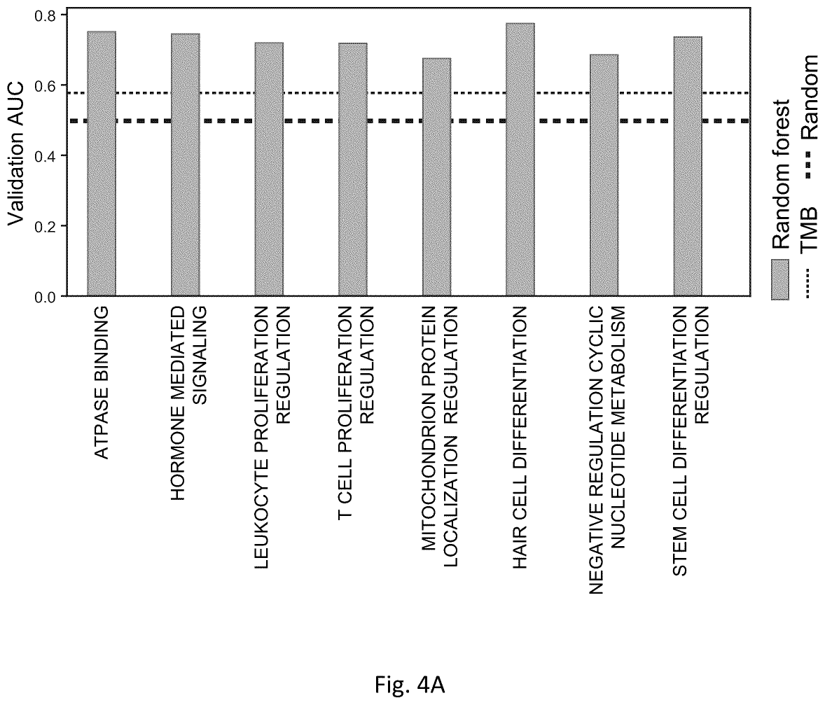

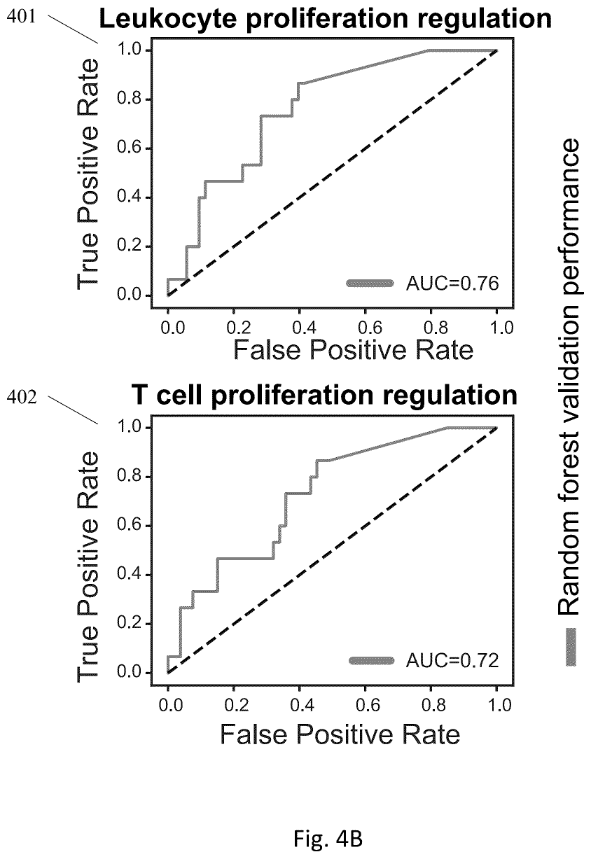

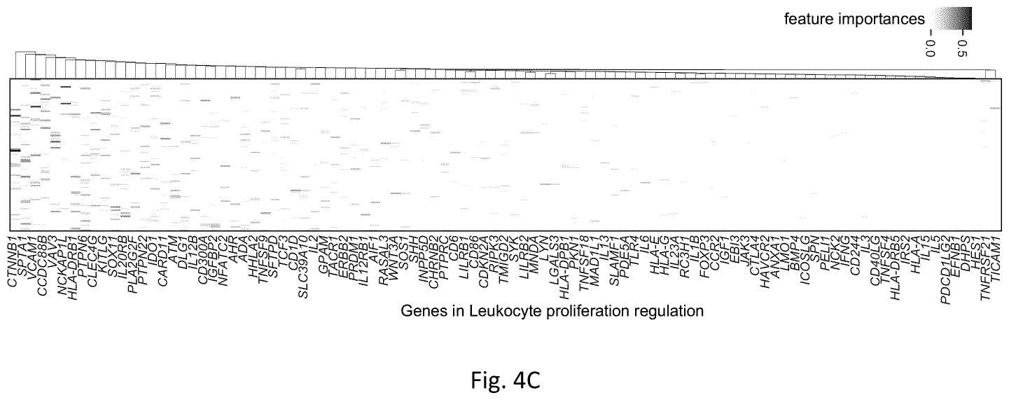

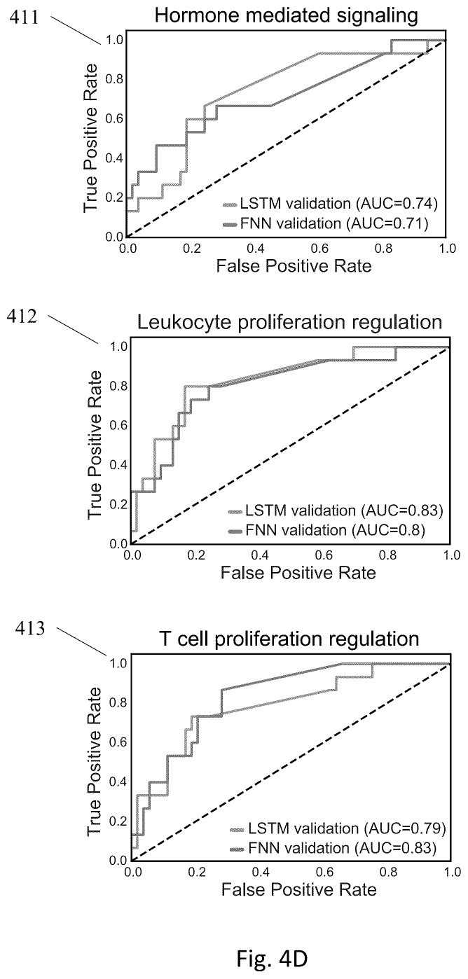

Greedy forward feature selection, that iteratively finds the best new feature to add to a set of selected features, was first investigated. In this process, the algorithm starts with an empty set, and then iterates over all genes in a biological process, to add the gene that best improves the predictive performance. When using the greedy forward selected genes within each biological process, several biological processes showed high predictive performance on the training dataset, (ROC AUC>0.75). However, none of these predictors maintained high performance in the validation dataset (that is, at least 90% of the training performance). It was therefore reasoned that greedy feature selection strategy impaired generalization by converging into local optimum. As such, randomized forward feature selection was employed, which sequentially selects features to add using a probabilistic function (see methods for details). In contrast to the greedy forward selector, four processes that performed well on the training dataset maintained high performance when applied to the validation dataset (FIG. 3B and FIG. 3C). These include RNA polymerase II transcription regulation, enzyme regulator activity, establishment of protein localization and regulatory regions of nucleic acid binding (FIG. 4B). Next, a genetic algorithm feature selection was applied (see Tan, F., et al., Soft Comput. 2008; Wang, L., et al., Methods, 2016; and Jagdhuber, R., et al., BMC Bioinformatics, 2020). This method outperformed the forward selection algorithms, where selected subsets of mutated genes in 15 processes maintained high performance on the validation dataset (FIG. 3B and FIG. 3C). The best performing processes include immune response, leukocyte differentiation and cell motility (FIG. 3B and FIG. 3C). Several genes that were frequently selected within these processes have important roles in melanoma progression and prognosis. These include CD44, shown to have an effect on tumor progression and subsequent poor prognosis (see Wu, R.-L. et al., Sci. Rep., 2018; and Dietrich, A., et al., Eur. J. Cancer Oxf. Engl., 1997) and TNFSF14, a regulator of T-cell proliferation that is commonly expressed in melanomas (see Mortarini, R. et al. Cancer Res., 2005).

Importantly, using all three feature selection methods, the biological processes with best performance on the training dataset performed significantly better on the validation dataset compared to processes that showed poor performance on the training dataset (FIG. 3D). A positive correlation was observed between the performances of selected subsets of mutated genes in different biological processes across the feature selection methods (FIG. 3E). Overall, these results support the premise that subsets of mutated genes within specific biological processes maintain comparable predictive performance to that of the TMB.

### Non-Linear Selection of Mutation Subsets in Biological Processes

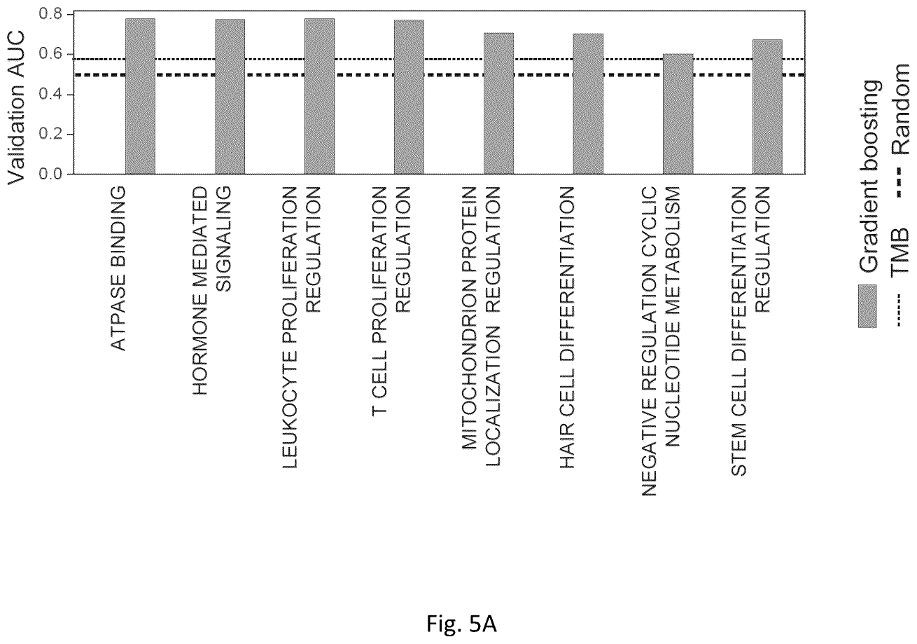

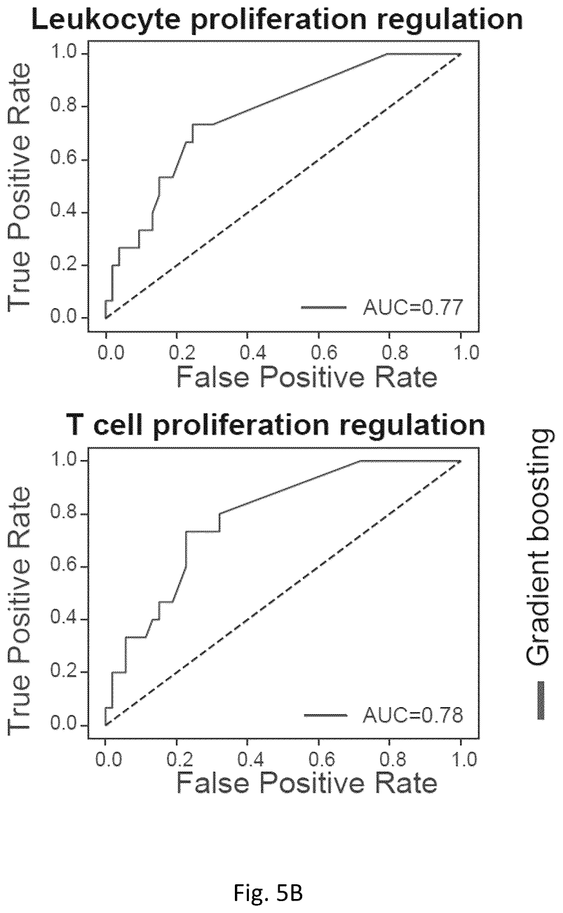

While using selected subsets of mutated genes indicates several top pathways are approximately equivalent to the TMB, none of the best-performing processes demonstrated a substantial improvement over the TMB. To obtain an ICI response predictor that outperforms the TMB based on tumor mutations, alternative classification techniques were examined. Accounting for complex interactions between mutated genes in biological processes may be critical for prediction of ICI response, therefore non-linear classifiers were applied to mutated genes within each biological process. First, trained decision tree algorithms were trained, including random forest (RF) and gradient boosting (GB) using mutations in all sequenced genes within a biological process. The top biological processes using both methods showed a strong predictive capability across the training and validation datasets (FIG. 4A, FIG. 5A, and FIG. 5B). In contrast to the sum of mutation classifiers, the top decision-trees predictors substantially exceeded TMB performance for the validation dataset (FIG. 4A, FIG. 5A and FIG. 5B). Interestingly, leukocyte proliferation regulation and T-cell proliferation regulation were among the top biological processes, both directly linked to ICI related immune responses; checkpoint inhibitor antibodies prevent T-cell inhibition and promotes the proliferation of effector T cells (see Darvin, P., et al., Exp. Mol. Med., 2018), and their response to these treatments require their proliferation and presence in the tumor microenvironment (see Jenkins, R. W., et al., Br. J. Cancer, 2018) (FIG. 4B). The mutated genes in the leukocyte proliferation regulation process with the highest contribution to the RF prediction capacity were investigated. Mutations in beta catenin gene CTNNB1 had the highest contribution for prediction, in agreement with recent findings that activation of this gene in melanoma cells is associated with a reduction in T-cell antitumor response (see Spranger, S., et al., Nature, 2015). In addition, among the top contributing genes in that process was IL2, a gene with known antitumor activity by increasing T-cell proliferation and previously used clinically to treat cancers (see Domingues, B., et al., ImmunoTargets Ther., 2018; and Agarwala, S. S., Expert Rev. Anticancer Ther., 2009), and CD137, another known target for antibody mediated immunotherapy target previously tested in clinical trials (see Yonezawa, A., et al., Clin. Cancer Res., 2015) (FIG. 4C). To further investigate non-linear predictors that may capture complex interactions between mutated genes within these processes, two classes of neural network models were evaluated using mutated genes within the top processes. Both the Forward Neural Network and Long Short-Term Memory Recurrent Neural Network models demonstrated high predictive capacity when applied to mutations within these biological processes (FIG. 4D).

### Validation of Clinical Utility in Sub-Optimal Conditions

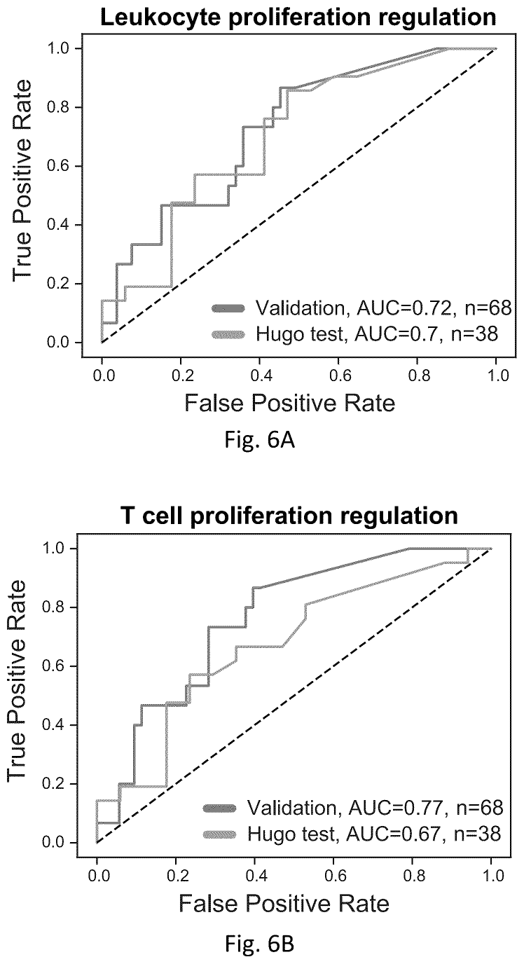

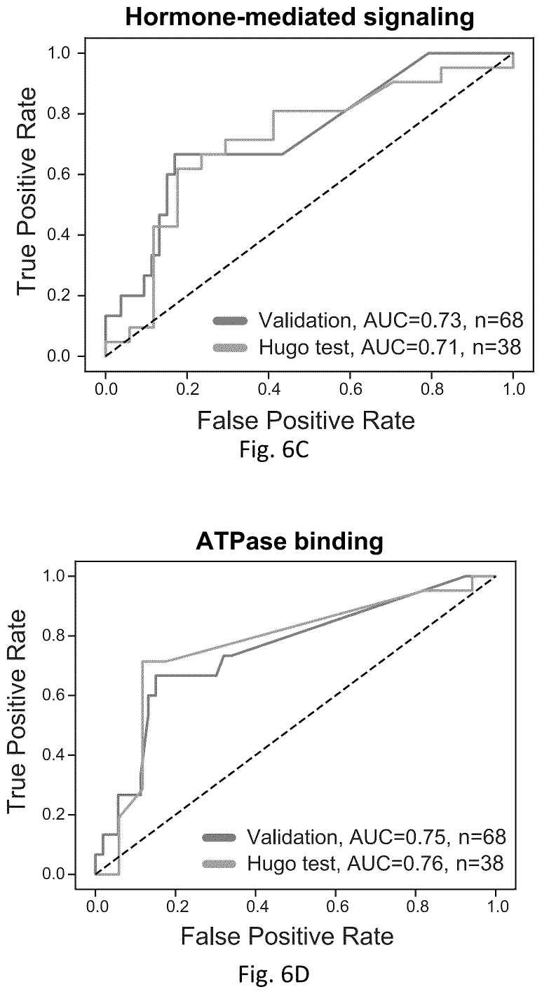

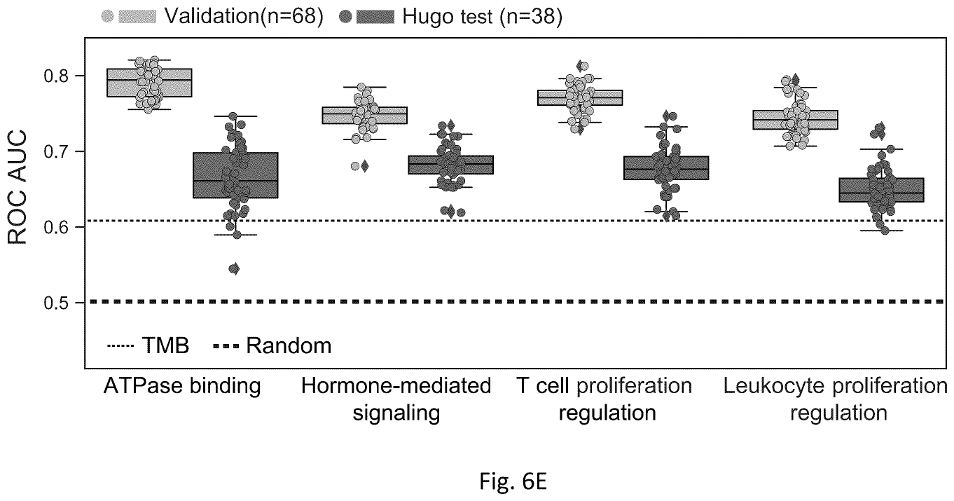

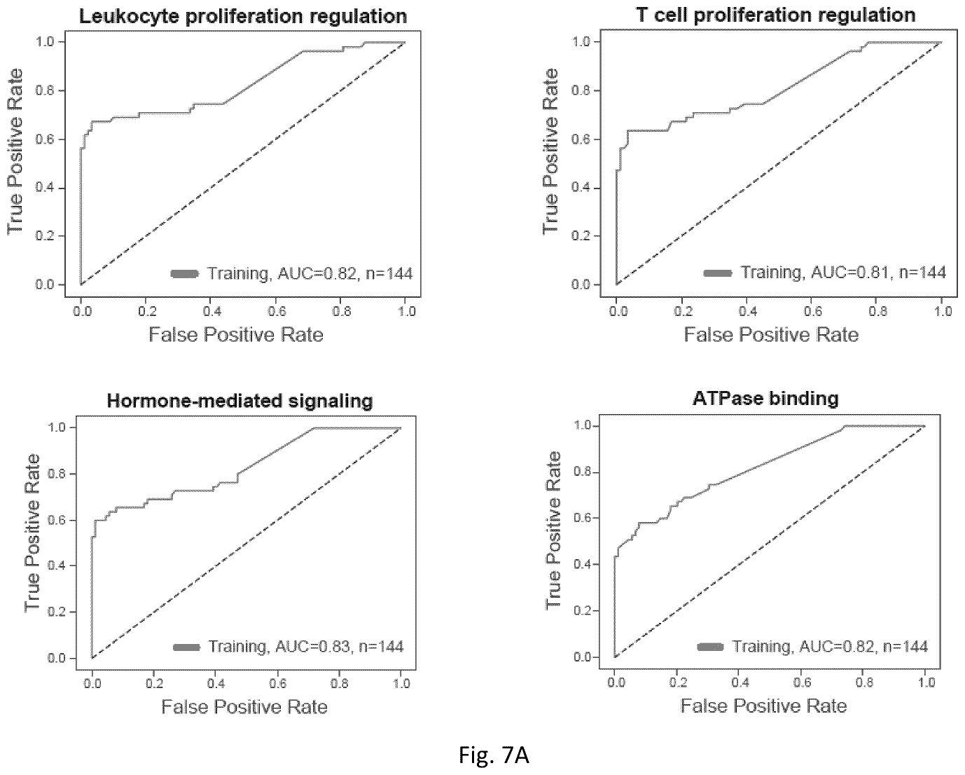

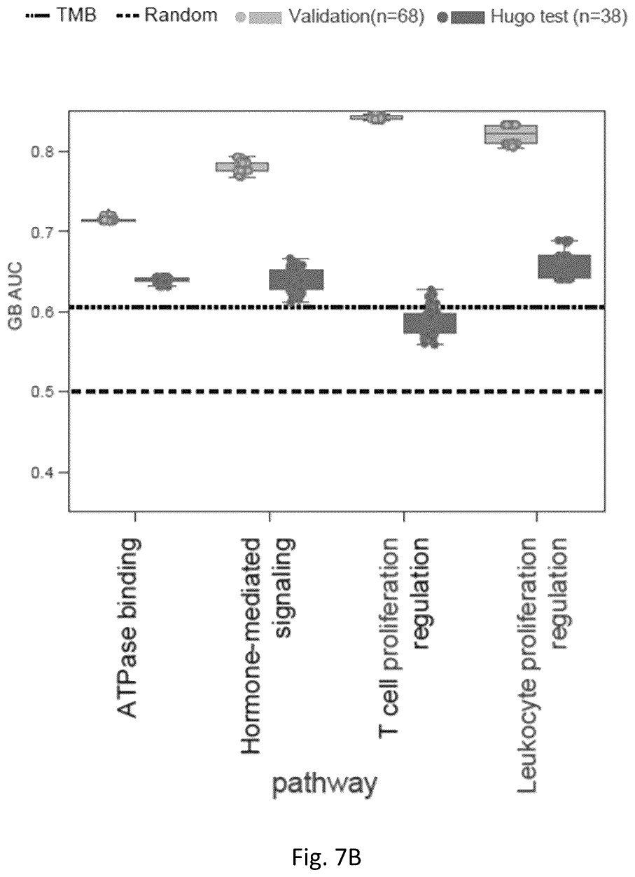

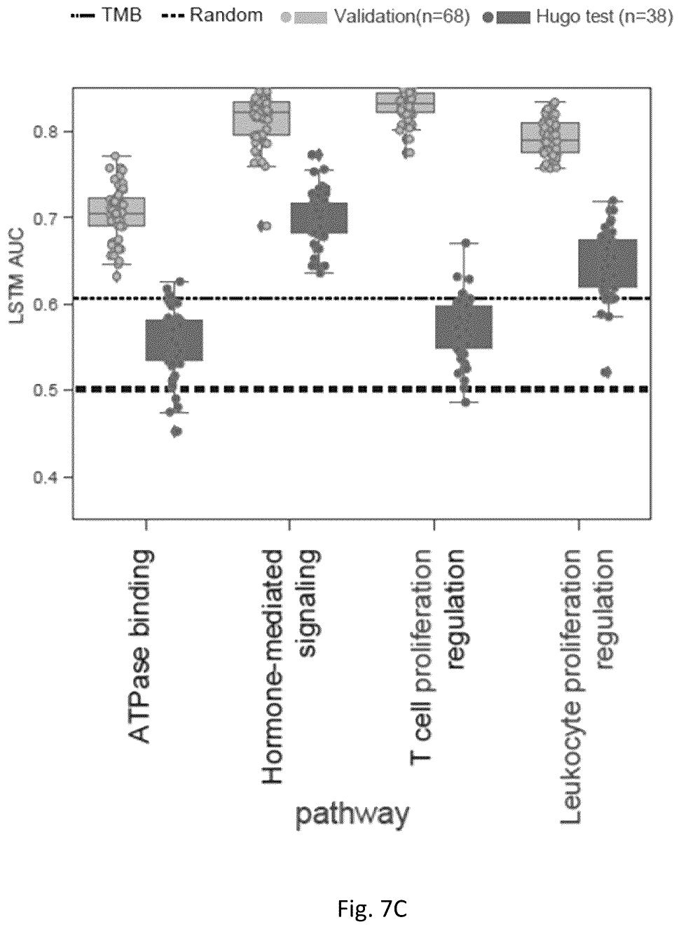

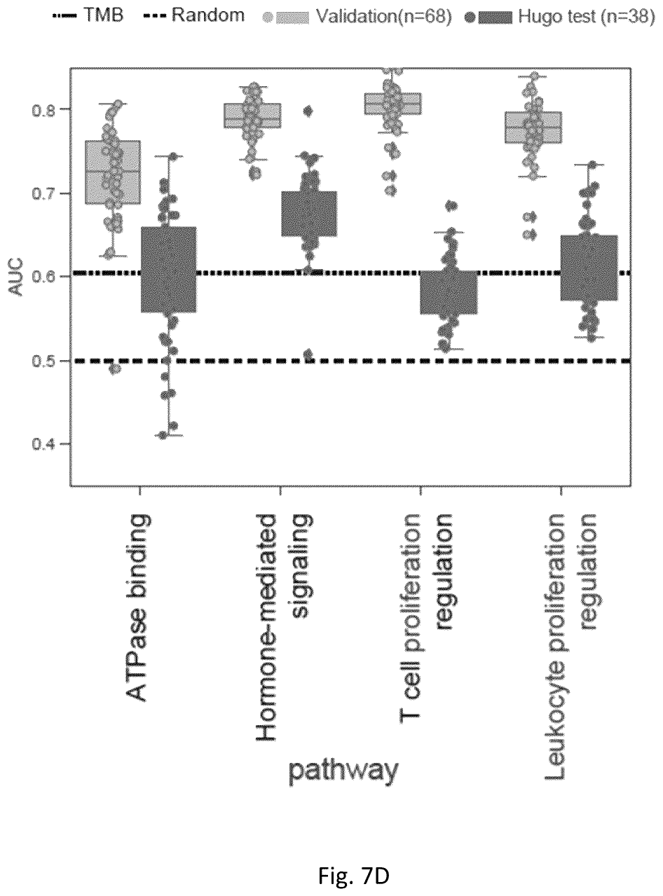

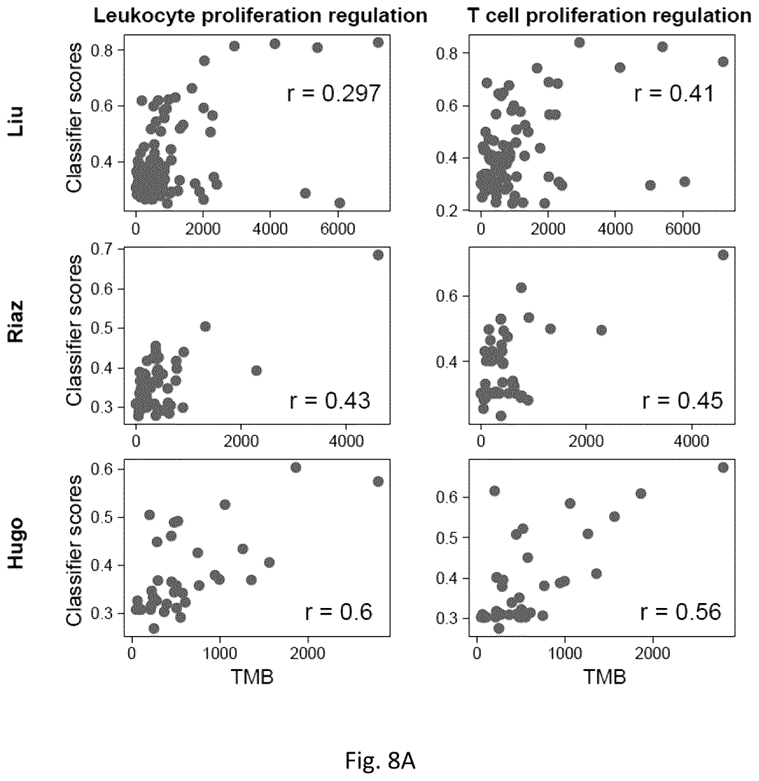

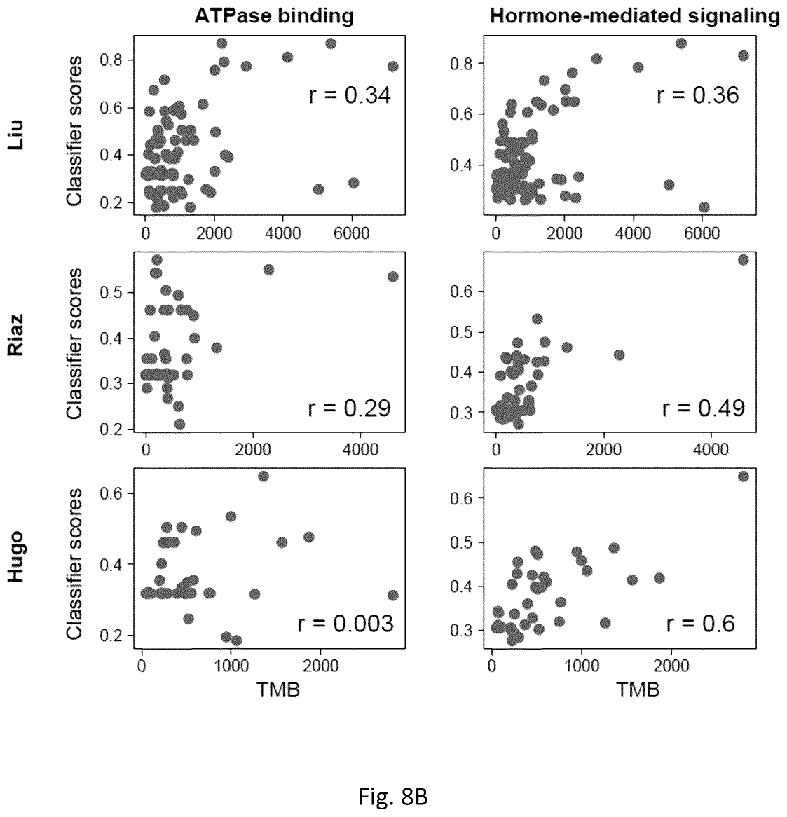

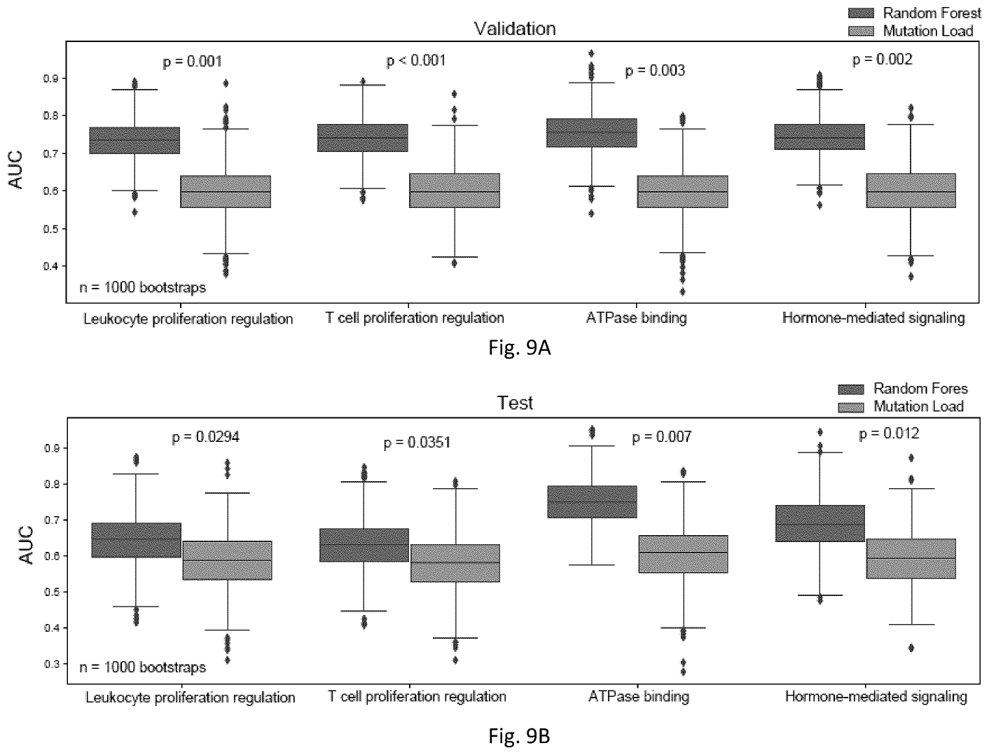

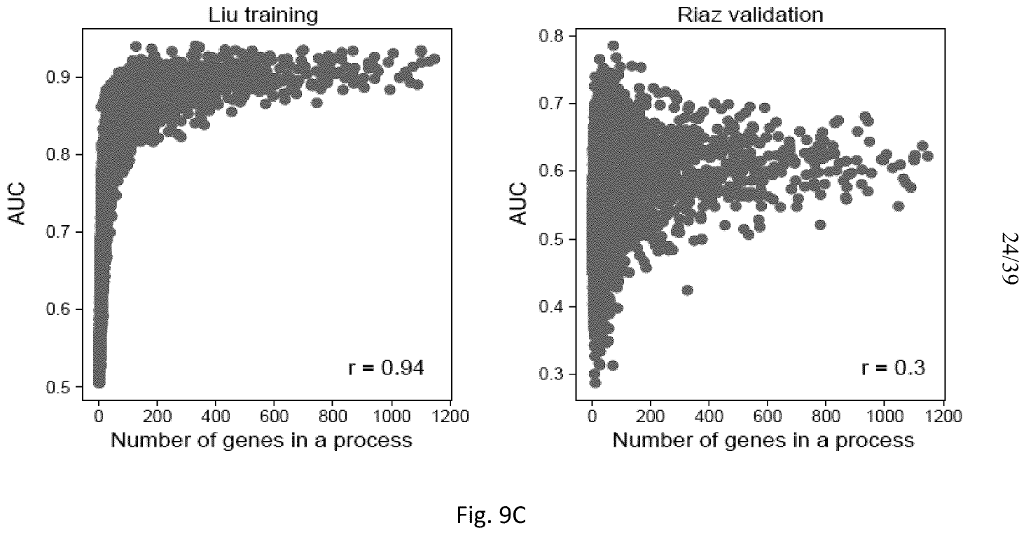

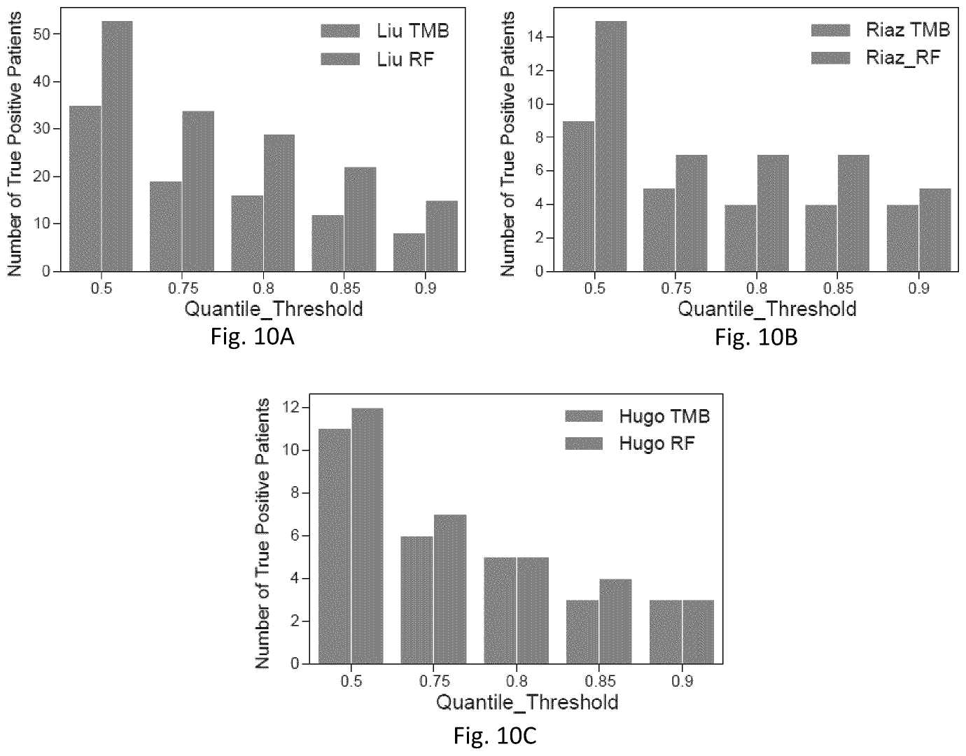

To test the potential clinical utility of the selected four biological process-based predictors, their performance was examined using an additional test dataset where not all genes used for training are sequenced. This dataset (see Hugo, W. et al., Cell, 2016) comprises mutation and response data from 38 melanoma patients treated with anti-PD1, but included only 59-68% of the genes used to train the classifiers. This data was unseen for the complete training and validation process, and only the selected classifiers that demonstrated high predictive performance in the validation dataset were evaluated in this dataset Remarkably, despite this, the process mutation RF classifiers maintained their high predictive performance for this dataset (FIG. 6A-6D and FIG. 7A-FIG. 7D). To test the robustness of this approach these classifiers were evaluated when retrained using different random seeds (see methods). This analysis revealed that the performance on both unseen datasets is maintained with the RF classifiers and is consistently better compared to TMB (FIG. 6E). Notably, RF classifiers were the most robust when presented with missing features in the test dataset (see Hugo, W. et al., Cell, 2016) (FIG. 7A-FIG. 7D). Importantly, there were only mild correlations between the overall TMB and the classification scores yielded by the RF predictors, supporting that these biological process based classifiers are capturing more than just an estimate of the TMB (FIG. 8A and FIG. 8B). Moreover, using a bootstrapping process, the top RF classifiers were found to perform significantly better than the TMB (FIG. 9A and FIG. 9B). As expected, the number of genes in a process strongly correlates with the RF predictor performance in the training dataset (by allowing more complex decision rules), however, there is only slight association between the number of process genes and predictor performance in the validation dataset (FIG. 9C). Further exploring this, using different classifier thresholds, more responding patients are correctly predicted with the leukocyte proliferation regulation RF predictor compared to the TMB (FIG. 10). As a result, some responding patients that are not captured by the TMB are predicted as responders by the leukocyte proliferation regulation RF classifier.

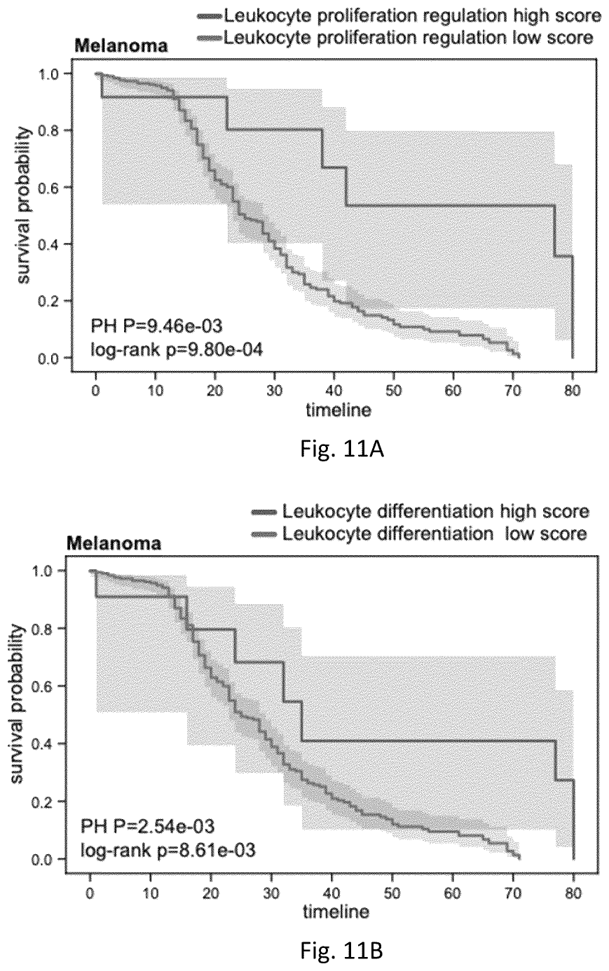

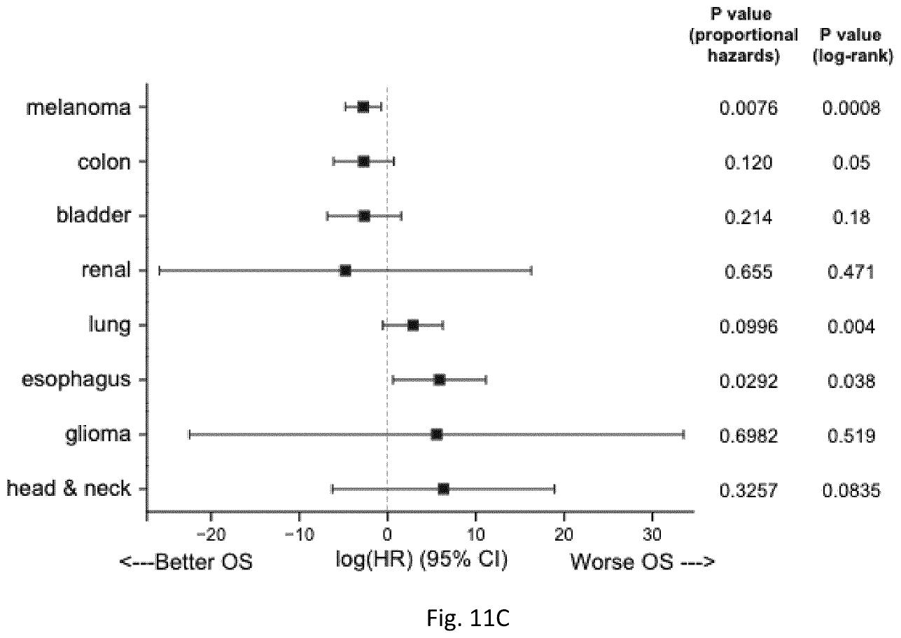

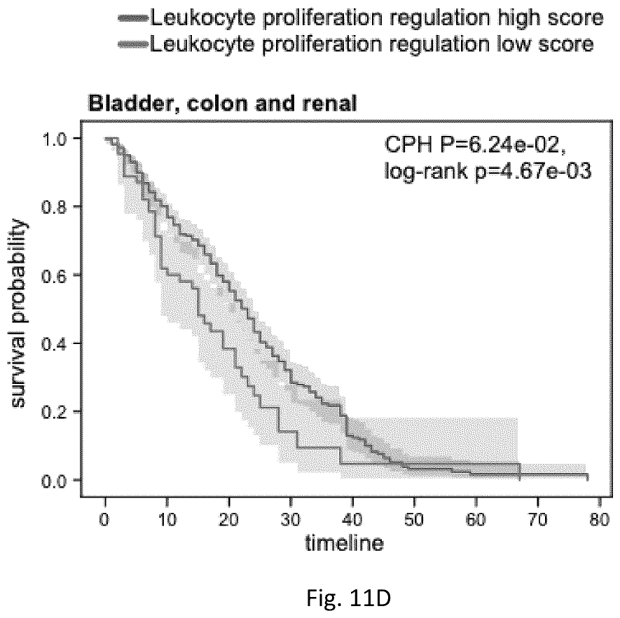

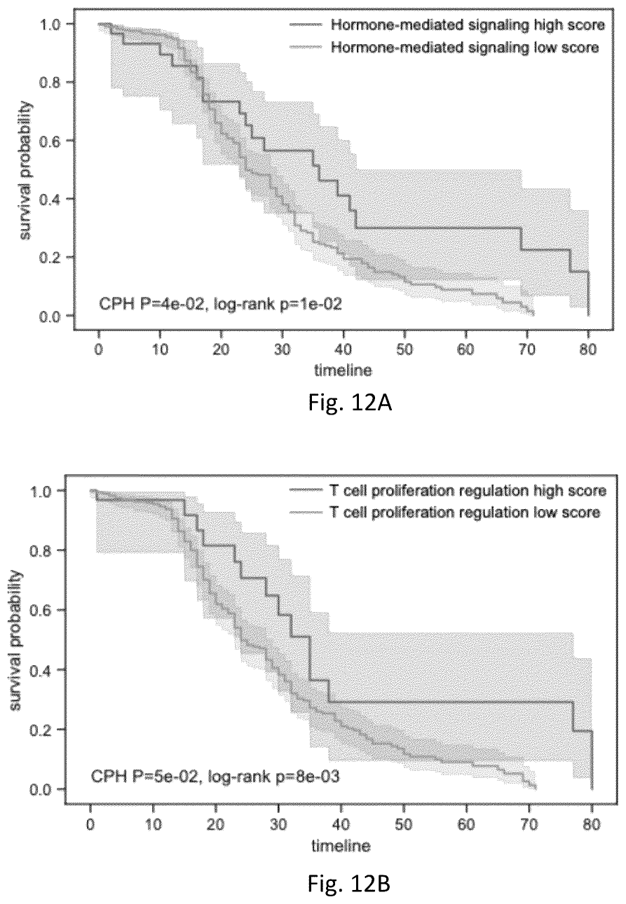

To further evaluate the potential clinical utility of these classifiers, their ability to predict overall survival was assessed in an independent dataset, the Memorial Sloan Kettering Cancer Center (MSKCC) data of patients treated with anti-PD1 (see Samstein, R. M. et al., Nat. Genet., 2019). This data was also kept unseen for the training and validation process and was used to test only the selected classifiers that demonstrated high predictive performance in the validation. This MSKCC dataset includes 321 melanoma patients treated with anti-PD1; this mutation data is limited to only 468 genes in the MSK-IMPACT targeted set. Nevertheless, the four RF mutated process models trained previously were significantly predictive of survival in this dataset, and in particular, the leukocyte proliferation regulation process was significant and strongly predictive (FIG. 11A, FIG. 12A, and FIG. 12B). Using the predictors based on sum of mutations and the genetic algorithm feature selection, a higher number of mutations in the leukocyte differentiation process was predictive of ICI response (FIG. 3B and FIG. 3C). The sum of mutations in selected genes in this process was also strongly predictive of overall survival in the MSKCC dataset (FIG. 11B).

### Analysis of Model Performance in Untrained Datasets

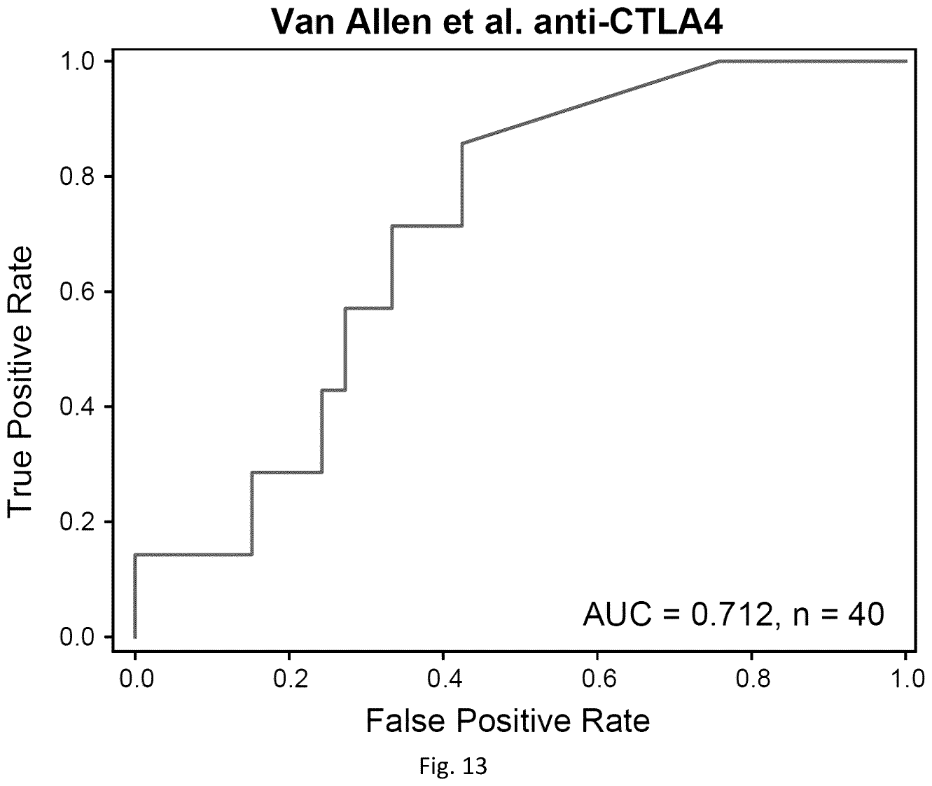

To evaluate the performance of the leukocyte proliferation regulation RF classifier in another treatment context, the model was applied, without further training, to predict response to CTLA4 inhibitor therapy through an independent dataset (see Van Allen, E. M. et al., Science, 2015). Even though it was trained to predict anti-PD1 response, the leukocyte proliferation regulation RF classifier was predictive of anti-CTLA4 response, demonstrating potential utility in a larger clinical context (FIG. 13).

It was then evaluated whether the leukocyte proliferation regulation RF classifier, which obtained the best performance over all datasets, may be applicable to other cancer types. To this end, it was applied to predict overall survival for other cancer types included in the MSKCC dataset. In addition to melanoma, three cancers (colon, bladder, and renal) showed positive association between the leukocyte proliferation regulation predictor and overall survival following anti-PD1 treatment (FIG. 11C). When pooling samples from the three non-melanoma cancer types together, the leukocyte proliferation regulation predictor demonstrated significant overall survival predictive capability via log-rank test (FIG. 11D).

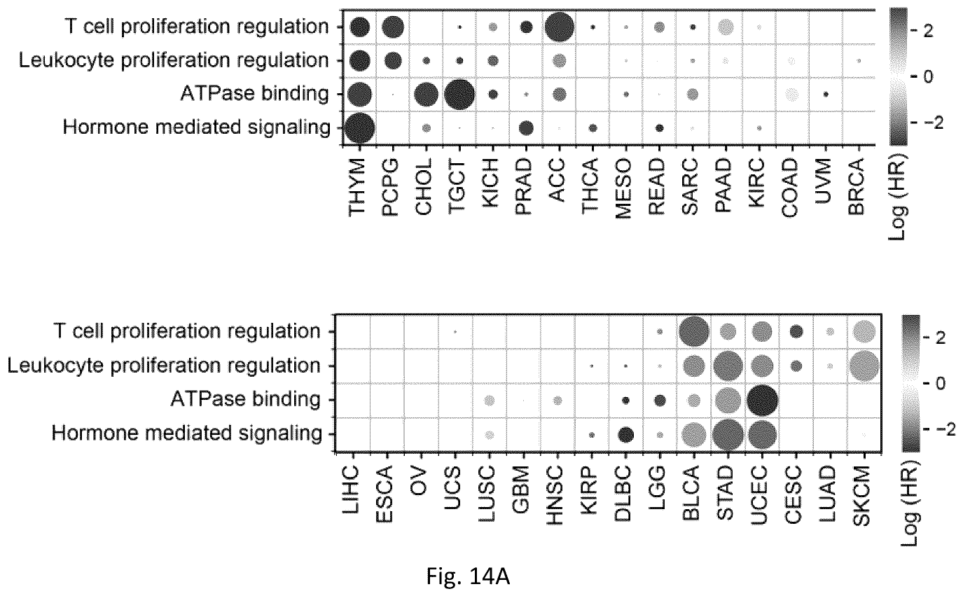

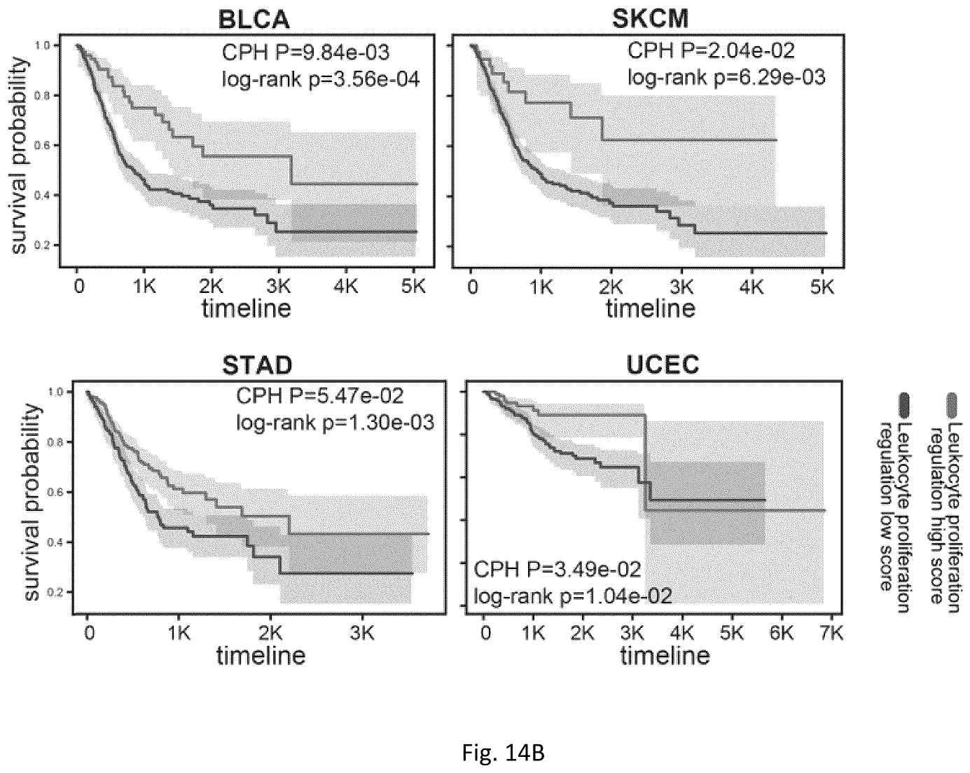

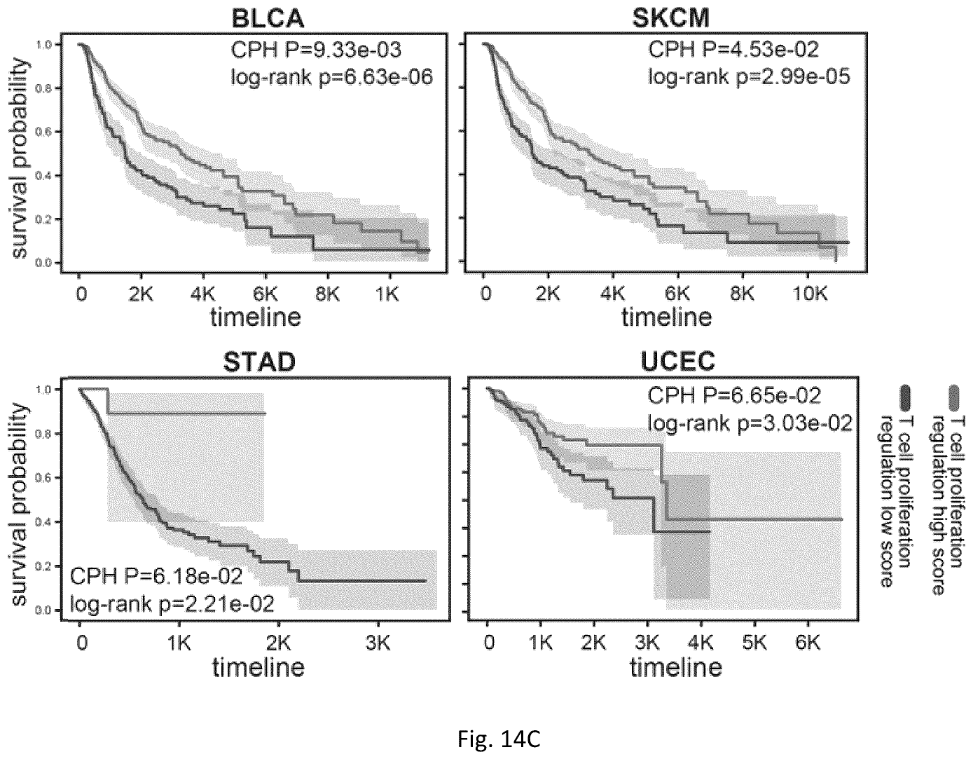

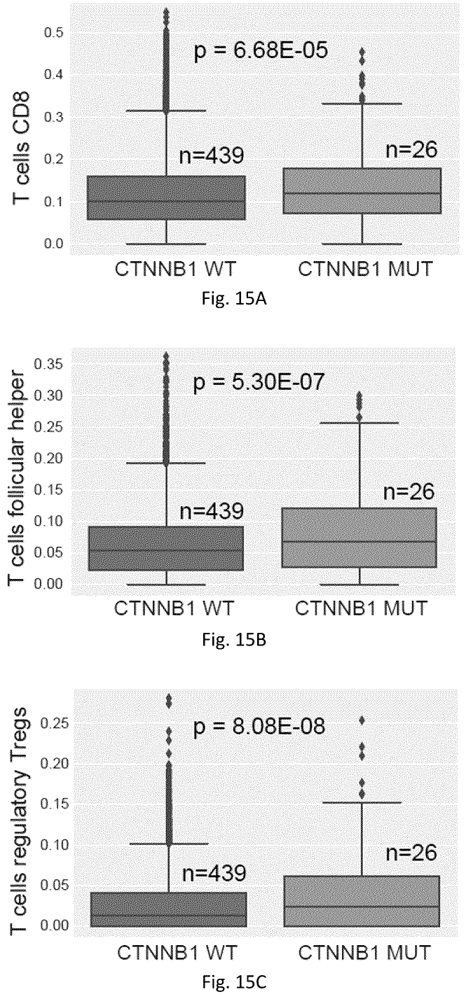

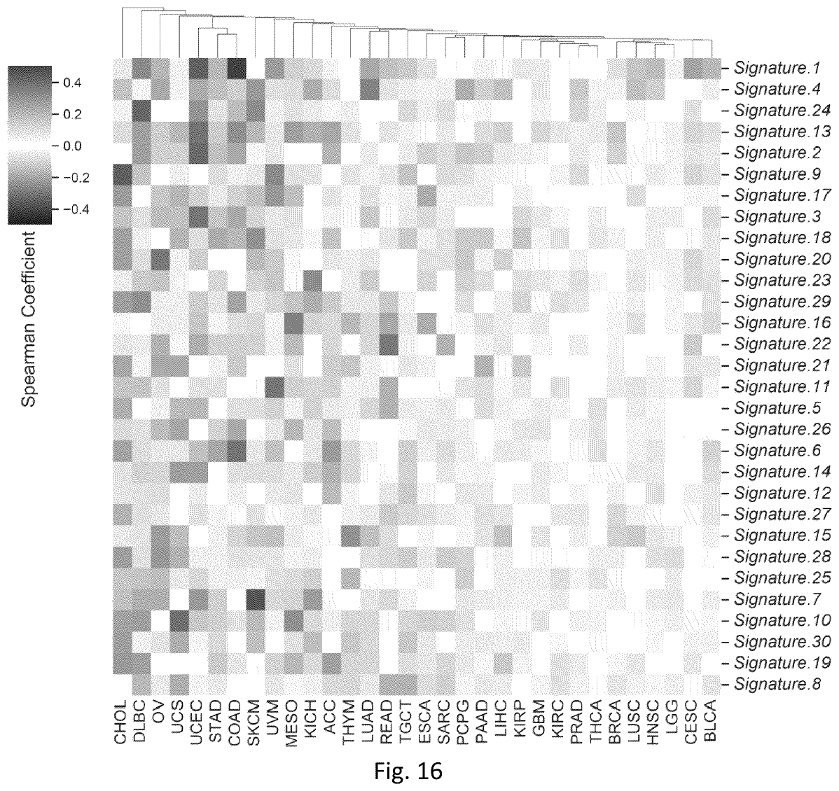

Finally, the prognostic value of the top RF predictors derived through this work was evaluated in different cancer types from The Cancer Genome Atlas (TCGA) dataset. To this end, the classifiers that were trained on the Liu data based on mutations within the four selected biological processes were applied to 32 cancer types from TCGA. Leukocyte and T cell proliferation regulation process RF classifiers were predictive of overall survival in SKCM, UCEC, STAD and BLCA (FIG. 14A-FIG. 14C). Importantly, for the latter three cancer types, all four RF process classifiers were significantly predictive of overall survival. The leukocyte proliferation regulation RF classifier was the strongest predictor of survival across TCGA cancer types. The analysis in FIG. 4C showed that beta-catenin gene, CTNNB1, contributes most to classification in the leukocyte proliferation regulation RF model. While CTNNB1 activation has been associated with immune exclusion in melanoma cells (see Spranger, S., et al., Nature, 2015), it may be associated with improved ICI responses on T cells. To better understand the context in which CTNNB1 contributes to prediction of ICI response, CIBERSORT (see Newman, A. M. et al., Nat. Methods, 2015) was applied to TCGA samples, and the association between CTNNB1 mutations and the predicted abundances of different immune cell types was investigated. Interestingly, different subsets of CIBERSORT inferred T-cells are significantly higher in CTNNB1 mutated melanoma tumors compared to wild type CTNNB1 tumors (FIG. 15A, FIG. 15B, and FIG. 15C). To better understand the association between the leukocyte proliferation regulation RF classifier with ICI response in different cancer types, the classifier scores were correlated with mutation signatures (see Alexandrov, L. B., et al., Cell, 2013) in different cancer types through TCGA (FIG. 16). In SKCM, the strongest correlation observed was with signature 7, which is linked with ultraviolet light exposure. Similarly, the strongest correlation in LUAD was found to be signature 4, linked with tobacco smoking, and the strongest association with COAD was signature 6, linked with defective mismatch repair.

B and T Cell Burden Scores do not Correlate with ICI Response

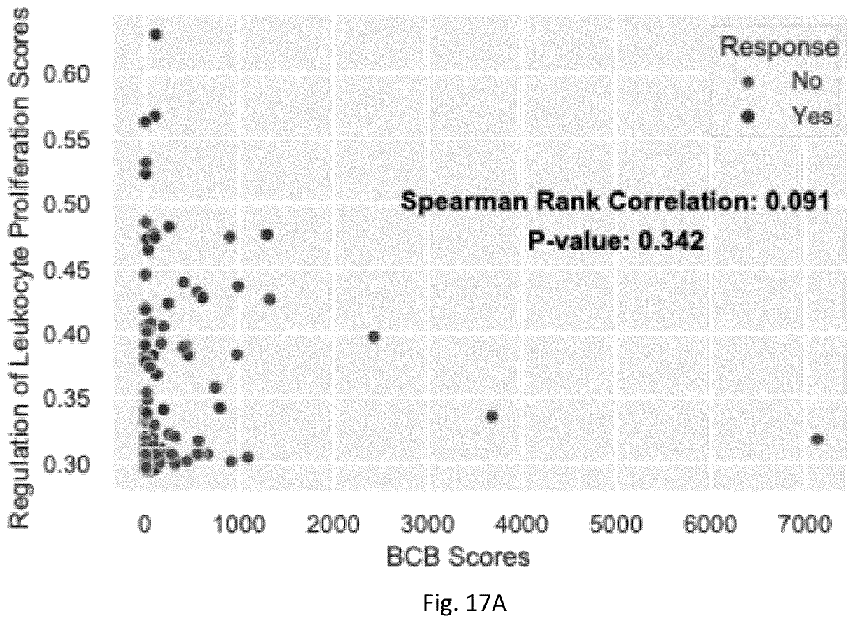

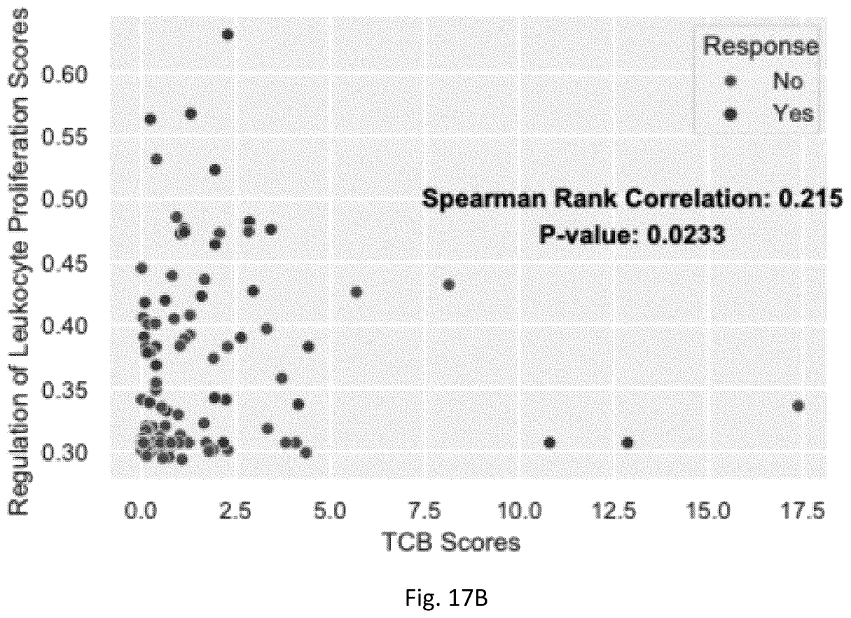

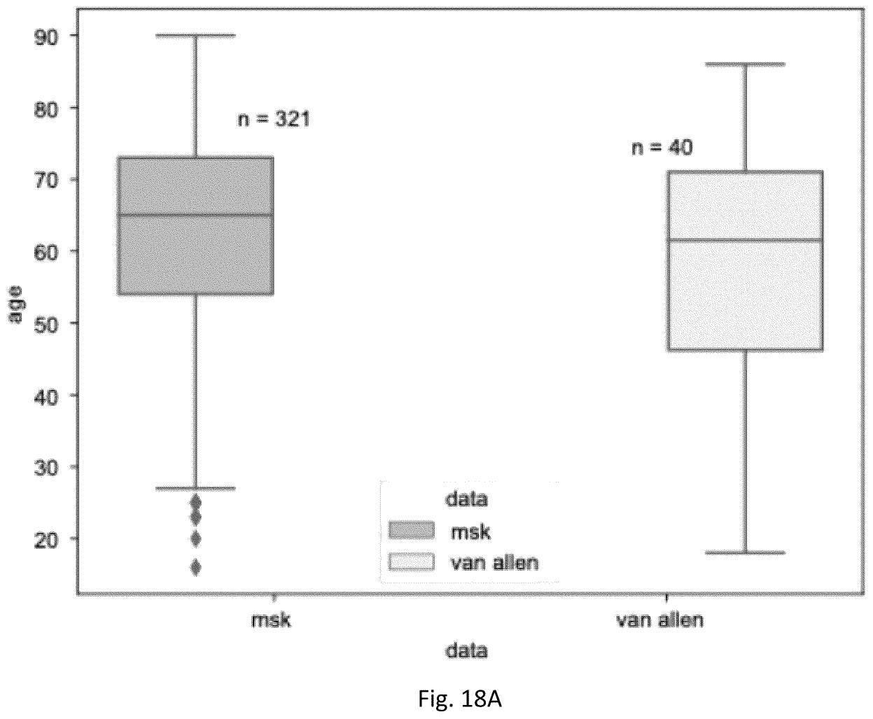

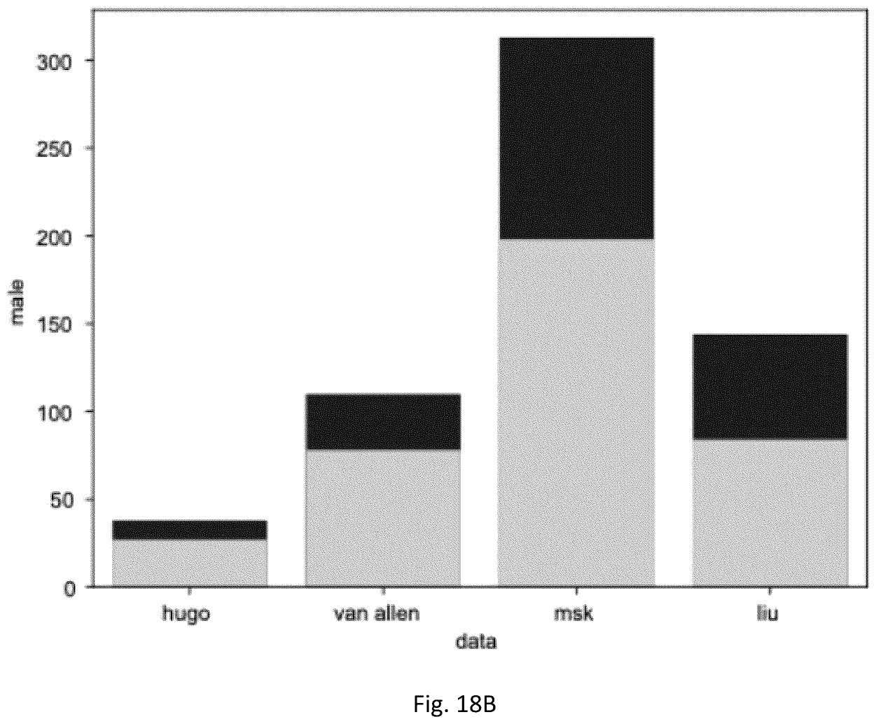

In further comparing the present results, only moderate correlation was observed between the leukocyte proliferation regulation classifier scores with B and T cell burden scores (BCB and TCB respectively) that have been published recently (Freeman, S. S. et al., Cell Rep. Med., 2022), supporting an independent prognostic value (FIG. 17A and FIG. 17B). In addition, patients with high BCB or TCB scores are not associated with increased response, as reported (Freeman, S. S. et al., Cell Rep. Med., 2022), whereas patients with high leukocyte proliferation regulation classifier scores are associated with response, supporting the potential clinical value of this classifier (FIG. 17A and FIG. 17B).

### Impacts of Predictive Modeling on ICI Therapy

Understanding the mechanisms underlying response and resistance to ICI therapy is critical to improving treatment of melanoma as well as other types of cancer. Through different feature selection and classification methods, analyzing tumor mutations in the context of biological processes enhances the predictive performance of ICI response compared to existing genomic predictors. Using feature selection methods, subsets of genes within distinct biological processes were identified in which the mutation burden presents an alternative biomarker to the genome-wide TMB. To further enhance the predictive performance, nonlinear classifiers were trained using mutated genes in distinct biological processes. Nonlinear classification methods have the potential to capture complex associations between ICI responses and mutated genes within a process. Using a random forest method substantially improves the predictive capability of predictors trained using mutations in specific processes, demonstrating significantly better performance compared to the TMB. Among the processes that maintain the best performance are leukocyte and T-cell proliferation regulation, known to play an important role in immune infiltration and ICI treatment. The predictive performance of these process classifiers is consistent across multiple datasets and remains stable across varying sequencing coverage.

Different methods to predict treatment benefit were investigated using mutations in the context of biological processes, which demonstrate several notable improvements over the TMB. First, the models in this work require substantially fewer genes to be sequenced for prediction. For example, the leukocyte proliferation regulation predictor requires sequencing of 99 genes, and the T-cell proliferation regulation predictor requires sequencing of 73 genes. It was also found that a smaller subset of genes within these processes would retain a similar predictive power. Less than 20 genes were sufficient to maintain a comparable performance, with the caveat that for this analysis, the performance was evaluated on the 3 datasets together. Second, developing biomarkers based on distinct biological processes improves their interpretability, and allows investigation of the mechanisms underlining their clinical utility. In particular, using non-linear classifiers substantially improves the predictive capability of mutated processes, by simultaneously accounting for mutations associated with either resistance or response to treatment.

More generally, somatic mutations within distinct immune and signaling processes have a strong predictive performance of ICI responses in melanoma. This finding suggests that interactions between tumor genetic alterations and the microenvironment underline, at least in part, ICI responses. This could be facilitated through altered antigen presentation, supported by several HLA mutations that are frequently selected in trees within the random forest classifier (FIG. 4C). Alternatively, or in complement, it is possible that mutated signaling processes modulate immune infiltration in the tumor microenvironment, supported by the selection of mutations in multiple signaling genes such as beta catenin and protein kinase and phosphatase genes (FIG. 4B and FIG. 6C). Supporting this notion, beta catenin mutations are associated with increased CIBERSORT-inferred abundances of different T cell subsets (FIG. 15A, FIG. 15B, and FIG. 15C).

Additionally, different processes were identified when using the mutation count classifiers than those identified with nonlinear classification methods. Interestingly, the leukocyte differentiation process was selected using the genetic algorithm feature selection, whereas the leukocyte proliferation regulation was selected using the decision tree algorithms. It is possible that while mutated leukocyte differentiation process is associated with ICI response, some of the mutated genes in the leukocyte proliferation regulation process may be associated with ICI resistance. Importantly, genes belonging to the leukocyte proliferation regulation process but not in the leukocyte differentiation process include several MHC class I complex genes (HLA-A, E, G, DRB1, DRB5 and DPB1), which are known to be associated with immune evasion and ICI resistance (see Dhatchinamoorthy, K., et al., Front. Immunol., 2021; and Lee, J. H. et al., Nat. Commun., 2020).

### Applicability to a Broader Range of Treatments

The methods implemented throughout this work may be applied to construct mutated process predictors of response to other treatments in different cancer types, as evidenced by the prognostic value demonstrated in the TCGA analysis.

This study also has several potential limitations that are important to discuss. First, despite the improved predictive performance of random forest classifiers, RF and similar methods are more complex and often less interpretable for clinical use. Nevertheless, this is not the first study demonstrating that non-linear classification methods can significantly improve prediction of ICI benefit (see Chowell, D. et al., Nat. Biotechnol., 2021). Incorporating clinical features to train random forest models may potentially further improve the performance obtained in this work, when data becomes available (Chowell, D. et al., Nat. Biotechnol., 2021). In addition, future developments may dissect the biological processes distinguished in this work to identify candidate targets to enhance treatment sensitivity. Second, similar to the TMB, the predictive models developed in this study account only for tumor factors and not for the tumor microenvironment. Third, it remains open to investigation whether the biological processes distinguished throughout this work for melanoma also determine ICI response in other types of cancer.

With further investigation and validation using additional data cohorts, the predictors developed throughout this work may present a compelling alternative to the tumor mutation burden for predicting patient response to ICI therapy.

The methods employed are described herein.

### Datasets

For training, 144 melanoma patients' samples were used from Liu et al (see Liu, D. et al., Nat. Med., 2019), including somatic mutations and anti-PD1 response information. For validation, 68 melanoma patients' samples with somatic mutations and clinical data from Riaz et al (see Riaz, N. et al., Cell, 2017) were used. To further test the models, 38 anti-PD1 treated melanoma patients' samples were used from Hugo, W. et al., Cell, 2016. For all datasets, responders were defined as patients with complete or partial response. Additionally, targeted mutation data and overall survival data was utilized from the MSKCC cohort (see Samstein, R. M. et al., Nat. Genet., 2019), including melanoma, colorectal, bladder, renal, lung, esophagus, glioma and head and neck cancers.

TCGA mutation data was downloaded from the Xena Browser (Goldman, M. J. et al., Nat. Biotechnol., 2020).

The processing of the WES cohorts is described in the original publication (see Hugo, W. et al., Cell, 2016; Samstein, R. M. et al., Nat. Genet., 2019; Freeman, S. S. et al., Cell Rep. Med., 2022; and Dhatchinamoorthy, K., et al., Front. Immunol., 2021). Briefly, these were processed using MuTect and Strelka for identification of small insertions or deletions. Generalization of a classifier to different cohorts across different processing methods is crucial to support its potential clinical utility. For further evaluation of the datasets, the sex and age distributions across the cohorts (whenever available) are provided in FIG. 16.

### Feature Selection for Biological Processes Mutation Load Predictors

Three feature selection methods were applied to mutations in genes belonging to each biological process, to select a subset of genes that best predict ICI response. To this end, the predictive performance is defined to be the resulting ROC AUC when using the number of mutations in selected genes in a process as scores, and the ICI response as labels. The following feature selection methods were applied to the training dataset:

Greedy Forward Selector. The greedy forward selection algorithm iteratively selects genes within a process that improves the predictive performance. The algorithm starts with an empty list of genes, and at each step, it adds to that list the gene (in a specific biological process) that results in the highest performance when added. For each biological process, a maximum of 10 iterations were run, where the stopping criteria was when 10 iterations were completed, or when none of the genes in a process improved the performance when added.

Probabilistic Forward Selector. The probabilistic forward selector algorithm is similar to the greedy forward selector, except that the selection of the gene to add in each step is randomized over a set of possible genes. The probability to add a gene that improves the performance when added was defined as

$$\frac{1}{number of total iterations + current iteration} .$$

Genetic Algorithm. The following steps of the Genetic Algorithm were applied to each biological process (a) Initialization of a population of size 20, where approximately 10% of the genes in the biological process were randomly selected for each instance in the initial population. (b) Evaluation of each instance in the population, where mutations in each gene set in the population were summed to predict ICI response. (c) The top half of the instances in the population, that is, those with the best predictive performance, were selected for reproduction, with randomly selected pairing. (d) Crossover was applied to the randomly selected pairs, until a population size of 20 was reached. 10 iterations of steps (b)-(d) were repeated, and the best solution was retained, corresponding to the sets of mutated genes that yielded the best performance predicting ICI response.

Decision Tree Predictors for Mutations within Different Biological Processes

Decision trees were trained to predict ICI response using the training dataset, where the classification scores obtained with these predictors were used to predict ICI response. The following algorithms were considered:

Random forest. Random Forest generates multiple decision trees from subsets of features of the data, which are ensembled into a single classifier, therefore reducing the risk of overfitting for large decision trees. RandomForestClassifier method from the sklearn.ensemble package was selected, with 100 estimators, a max depth of 5 and a minimum sample split of 2. Other parameters were defined to default.

Gradient Boosting. Gradient uses boosting to integrate relatively shallow decision trees and ensemble a set of weak learners into a single strong learner. The GradientBoostingClassifier method from the sklearn.ensemble package was used, with 100 estimators, a max depth of 2, a learning rate of 0.1, and the deviance loss function. All other parameters defined to default.

For reproducibility, the random state was set 100 throughout this work, except for the robustness analysis.

When testing on datasets with missing values (where some of the genes were not sequenced) the decision tree classifiers were retrained on the training dataset with the original random seed, for the subset of genes present in the new data.

Neural Network Predictors for Mutations within Different Biological Processes

Two neural network architectures were trained to predict ICI response, where the resulting classification scores were used for prediction. These include:

Feed Forward Neural Network, using one fully connected hidden layer with 5 hidden units and sigmoid activation.

Long Short-Term Memory (LSTM) recurrent neural networks, using one LSTM cell with five hidden units.

All neural networks were trained with tensorflow.keras, using Adam optimizer, with 100 epochs and a batch size of 27.

### Robustness Analysis

To evaluate the robustness of different methods, the classifiers were retrained using the mutations within the selected processes and evaluated the performance of 50 retrained classifiers for each selected process.

### Survival Analysis

Survival analysis was performed using the proportional hazards, using python lifelines.statistics package. Either the sum of mutations per process (genetic algorithm and forward feature selection) or the classification scores (decision trees and neural networks) were used for prediction. All results were evaluated when controlling for age and sex as confounders and stratified for different cancer types in analyses aggregating patients with different cancer types.

### Bootstrapping Analysis

To evaluate the significance at which the random forest classifiers outperform the TMB in predicting ICI response, based on the four processes selected in training, a bootstrap analysis was performed. Seventy-five percent of each cohort was down-sized 1000 times, each of the four top RF classifiers were applied to the down-sampled cohort, as well as the TMB, to obtain the prediction AUCs. The fraction of AUCs from the down-sampling procedure in which the TMB outperformed the RF classifiers was used as a permutation p-value.

### Down-Sampling Analysis

To evaluate the smallest subsets of genes that retain the predictive capability of the full set of genes in a process, genes were randomly subsampled from each of the four processes previously selected in training. For each run, 15-85% of the genes were subsampled and used to train an RF model for each pathway. This was run 10000 times for each pathway to determine the smallest subsets of genes which still retained predictive power across the datasets from Liu, Riaz, and Hugo comparable to the previously generated models (>0.7 ROC Score).

### REFERENCES

The following publications are incorporated herein by reference in their entirety.
American Cancer Society, “Cancer Facts & Figures 2021”, Atlanta: American Cancer Society; 2021.
American Cancer Society, “Cancer Facts & Figures 2017”, Atlanta: American Cancer Society; 2017.
American Cancer Society, “Survival Rates for Melanoma Skin Cancer”, Melanoma Skin Cancer Early Detection, Diagnosis, and Staging; 2021.
BHATIA, S., TYKODI, S. S. & THOMPSON, J. A. Treatment of Metastatic Melanoma: An Overview. Oncol. Williston Park N 23, 488-496 (2009).
Domingues, B., Lopes, J. M., Soares, P. & Pópulo, H. Melanoma treatment in review. ImmunoTargets Ther. 7, 35-49 (2018).
Jardim, D. L., Goodman, A., de Melo Gagliato, D. & Kurzrock, R. The Challenges of Tumor Mutational Burden as an Immunotherapy Biomarker. Cancer Cell 39, 154-173 (2021).
Sharma, P. & Allison, J. P. The future of immune checkpoint therapy. Science 348, 56-61 (2015).
Villanueva, J. et al. Acquired Resistance to BRAF Inhibitors Mediated by a RAF Kinase Switch in Melanoma Can Be Overcome by Cotargeting MEK and IGF-1R/PI3K. Cancer Cell 18, 683-695 (2010).
Larkin, J. et al. Combined Nivolumab and Ipilimumab or Monotherapy in Untreated Melanoma. N. Engl. J. Med. 373, 23-34 (2015).
Gide, T. N., Wilmott, J. S., Scolyer, R. A. & Long, G. V. Primary and Acquired Resistance to Immune Checkpoint Inhibitors in Metastatic Melanoma. Clin. Cancer Res. 24, 1260-1270 (2018).
Hodi, F. S. et al. Improved survival with ipilimumab in patients with metastatic melanoma. N. Engl. J. Med. 363, 711-723 (2010).
Robert, C. et al. Ipilimumab plus dacarbazine for previously untreated metastatic melanoma. N. Engl. J. Med. 364, 2517-2526 (2011).
Friedlaender, A., Addeo, A. & Banna, G. New emerging targets in cancer immunotherapy: the role of TIM3. ESMO Open 4, e000497 (2019).
Schadendorf, D. et al. Pooled Analysis of Long-Term Survival Data From Phase II and Phase III Trials of Ipilimumab in Unresectable or Metastatic Melanoma. J. Clin. Oncol. Off. J. Am. Soc. Clin. Oncol. 33, 1889-1894 (2015).
Wolchok, J. D. et al. Nivolumab plus ipilimumab in advanced melanoma. N. Engl. J. Med. 369, 122-133 (2013).
Gibney, G. T., Weiner, L. M. & Atkins, M. B. Predictive biomarkers for checkpoint inhibitor-based immunotherapy. Lancet Oncol. 17, e542-e551 (2016).
Chen, L. et al. CD38-mediated immunosuppression as a mechanism of tumor cell escape from PD-1/PD-L1 blockade. Cancer Discov. 8, 1156-1175 (2018).
Holderried, T. A. W. et al. Molecular and immune correlates of TIM-3 (HAVCR2) and galectin 9 (LGALS9) mRNA expression and DNA methylation in melanoma. Clin. Epigenetics 11, 161 (2019).
House, I. G. et al. Macrophage-Derived CXCL9 and CXCL10 Are Required for Antitumor Immune Responses Following Immune Checkpoint Blockade. Clin. Cancer Res. Off. J. Am. Assoc. Cancer Res. 26, 487-504 (2020).
Rooney, M. S., Shukla, S. A., Wu, C. J., Getz, G. & Hacohen, N. Molecular and genetic properties of tumors associated with local immune cytolytic activity. Cell 160, 48-61 (2015).
Hugo, W. et al. Genomic and Transcriptomic Features of Response to Anti-PD-1 Therapy in Metastatic Melanoma. Cell 165, 35-44 (2016).
Jiang, P. et al. Signatures of T cell dysfunction and exclusion predict cancer immunotherapy response. Nat. Med. 24, 1550-1558 (2018).
Auslander, N. et al. Robust prediction of response to immune checkpoint blockade therapy in metastatic melanoma. Nat. Med. 24, 1545-1549 (2018).
Charoentong, P. et al. Pan-cancer Immunogenomic Analyses Reveal Genotype-Immunophenotype Relationships and Predictors of Response to Checkpoint Blockade. Cell Rep. 18, 248-262 (2017).
Pérez-Guijarro, E. et al. Multimodel preclinical platform predicts clinical response of melanoma to immunotherapy. Nat. Med. 26, 781-791 (2020).
Du, K. et al. Pathway signatures derived from on-treatment tumor specimens predict response to anti-PD1 blockade in metastatic melanoma. Nat. Commun. 12, 6023 (2021).
Litchfield, K. et al. Meta-analysis of tumor- and T cell-intrinsic mechanisms of sensitization to checkpoint inhibition. Cell 184, 596-614.e14 (2021).
Byron, S. A., Van Keuren-Jensen, K. R., Engelthaler, D. M., Carpten, J. D. & Craig, D. W. Translating RNA sequencing into clinical diagnostics: opportunities and challenges. Nat. Rev. Genet. 17, 257-271 (2016).
Research, C. for D. E. and. FDA grants accelerated approval to pembrolizumab for first tissue/site agnostic indication. FDA (2019).
Research, C. for D. E. and. FDA approves pembrolizumab for adults and children with TMB-H solid tumors. FDA (2020).
Xuan, J., Yu, Y., Qing, T., Guo, L. & Shi, L. Next-generation sequencing in the clinic: Promises and challenges. Cancer Lett. 340, 284-295 (2013).
Galluzzi, L., Spranger, S., Fuchs, E. & López-Soto, A. WNT Signaling in Cancer Immunosurveillance. Trends Cell Biol. 29, 44-65 (2019).
Paschen, A., Melero, I. & Ribas, A. Central Role of the Antigen-Presentation and Interferon-γ Pathways in Resistance to Immune Checkpoint Blockade. Annu. Rev. Cancer Biol. 6, null (2022).
The Gene Ontology Consortium. Gene Ontology Consortium: going forward. Nucleic Acids Res. 43, D1049-D1056 (2015).
The Gene Ontology Consortium. The Gene Ontology Resource: 20 years and still GOing strong. Nucleic Acids Res. 47, D330-D338 (2019).
Tan, F., Fu, X., Zhang, Y. & Bourgeois, A. G. A genetic algorithm-based method for feature subset selection. Soft Comput. 12, 111-120 (2008).
Wang, L., Wang, Y. & Chang, Q. Feature selection methods for big data bioinformatics: A survey from the search perspective. Methods 111, 21-31 (2016).
Jagdhuber, R., Lang, M., Stenzl, A., Neuhaus, J. & Rahnenführer, J. Cost-Constrained feature selection in binary classification: adaptations for greedy forward selection and genetic algorithms. BMC Bioinformatics 21, 26 (2020).
Wu, R.-L. et al. Hyaluronic acid-CD44 interactions promote BMP4/7-dependent Id1/3 expression in melanoma cells. Sci. Rep. 8, 14913 (2018).
Dietrich, A., Tanczos, E., Vanscheidt, W., Schöpf, E. & Simon, J. C. High CD44 surface expression on primary tumours of malignant melanoma correlates with increased metastatic risk and reduced survival. Eur. J. Cancer Oxf. Engl. 1990 33, 926-930 (1997).
Mortarini, R. et al. Constitutive expression and costimulatory function of LIGHT/TNFSF14 on human melanoma cells and melanoma-derived microvesicles. Cancer Res. 65, 3428-3436 (2005).
Darvin, P., Toor, S. M., Sasidharan Nair, V. & Elkord, E. Immune checkpoint inhibitors: recent progress and potential biomarkers. Exp. Mol. Med. 50, 1-11 (2018).
Jenkins, R. W., Barbie, D. A. & Flaherty, K. T. Mechanisms of resistance to immune checkpoint inhibitors. Br. J. Cancer 118, 9-16 (2018).
Spranger, S., Bao, R. & Gajewski, T. F. Melanoma-intrinsic β-catenin signalling prevents anti-tumour immunity. Nature 523, 231-235 (2015).
Agarwala, S. S. Current systemic therapy for metastatic melanoma. Expert Rev. Anticancer Ther. 9, 587-595 (2009).
Yonezawa, A., Dutt, S., Chester, C., Kim, J. & Kohrt, H. E. Boosting Cancer Immunotherapy with Anti-CD137 Antibody Therapy. Clin. Cancer Res. 21, 3113-3120 (2015).
Samstein, R. M. et al. Tumor mutational load predicts survival after immunotherapy across multiple cancer types. Nat. Genet. 51, 202-206 (2019).
Van Allen, E. M. et al. Genomic correlates of response to CTLA-4 blockade in metastatic melanoma. Science 350, 207-211 (2015).
Newman, A. M. et al. Robust enumeration of cell subsets from tissue expression profiles. Nat. Methods 12, 453-457 (2015).
Alexandrov, L. B., Nik-Zainal, S., Wedge, D. C., Campbell, P. J. & Stratton, M. R. Deciphering Signatures of Mutational Processes Operative in Human Cancer. Cell Rep. 3, 246-259 (2013).
Freeman, S. S. et al. Combined tumor and immune signals from genomes or transcriptomes predict outcomes of checkpoint inhibition in melanoma. Cell Rep. Med. 3, 100500 (2022).
Dhatchinamoorthy, K., Colbert, J. D. & Rock, K. L. Cancer Immune Evasion Through Loss of MHC Class I Antigen Presentation. Front. Immunol. 12, 469 (2021).
Lee, J. H. et al. Transcriptional downregulation of MHC class I and melanoma de-differentiation in resistance to PD-1 inhibition. Nat. Commun. 11, 1897 (2020).
Chowell, D. et al. Improved prediction of immune checkpoint blockade efficacy across multiple cancer types. Nat. Biotechnol. 1-8 (2021) doi:10.1038/s41587-021-01070-8.
Liu, D. et al. Integrative molecular and clinical modeling of clinical outcomes to PD1 blockade in patients with metastatic melanoma. Nat. Med. 25, 1916-1927 (2019).
Riaz, N. et al. Tumor and Microenvironment Evolution during Immunotherapy with Nivolumab. Cell 171, 934-949.e16 (2017).
Goldman, M. J. et al. Visualizing and interpreting cancer genomics data via the Xena platform. Nat. Biotechnol. 38, 675-678 (2020).

1. A method of predicting a response to a medical treatment in a subject, comprising the steps of:
selecting a set of biological processes;
selecting a training dataset and a validation dataset, each dataset comprising a set of genome data and clinical outcomes;
grouping a set of mutations into groups each corresponding to a biological process of the set of biological processes;
generating a set of classifiers, each comprising a combination of mutations, to predict a clinical outcome from one of the groups of mutations;
training the set of classifiers on the training dataset;
calculating, with the validation dataset, a performance level of each classifier in the set of classifiers;
calculating, on a test dataset comprising genome data of a subject, a predicted clinical outcome from a medical treatment on a subject based on a subset of the set of classifiers having a high performance level on the validation dataset; and
treating the subject based on the predicted response to the medical treatment.

2. The method of claim 1, wherein the step of generating the set of classifiers comprises a Greedy forward feature selection algorithm.

3. The method of claim 1, wherein the step of generating the set of classifiers comprises a randomized forward feature selection algorithm.

4. The method of claim 1, wherein the step of generating the set of classifiers comprises a genetic algorithm.

5. The method of claim 1, wherein the step of generating the set of classifiers comprises a random forest algorithm.

6. The method of claim 1, wherein the step of generating the set of classifiers comprise a gradient boosted tree.

7. The method of claim 1, wherein at least one classifier of the set of classifiers comprises a Forward Neural Network model.

8. The method of claim 1, wherein at least one classifier of the set of classifiers comprises a Long Short-Term Memory Recurrent Neural Network model.

9. A system for predicting a response to a medical treatment in a subject, comprising a non-transitory computer-readable medium with instructions stored thereon, which when executed by a processor perform steps comprising:
selecting a set of biological processes from a database of biological processes;
storing a training dataset and a validation dataset on the non-transitory computer-readable medium, each dataset comprising a set of genome data and clinical outcomes;
grouping a set of mutations into groups each corresponding to a biological process of the set of biological processes;
generating a set of classifiers, each comprising a combination of mutations, to predict a clinical outcome from one of the groups of mutations;
training the set of classifiers on the training dataset;
calculating, with the validation dataset, a performance level of each classifier in the set of classifiers;
calculating, on a test dataset comprising genome data of a subject, a predicted clinical outcome from a medical treatment on a subject based on a subset of the set of classifiers having a high performance level on the validation dataset; and
treating the subject based on the predicted response to the medical treatment.

10. The method of claim 9, wherein the step of generating the set of classifiers comprises a Greedy forward feature selection algorithm.

11. The method of claim 9, wherein the step of generating the set of classifiers comprises a randomized forward feature selection algorithm.

12. The method of claim 9, wherein the step of generating the set of classifiers comprises a genetic algorithm.

13. The method of claim 9, wherein the step of generating the set of classifiers comprises a random forest algorithm.

14. The method of claim 9, wherein the step of generating the set of classifiers comprise a gradient boosted tree.

15. The method of claim 9, wherein at least one classifier of the set of classifiers comprises a Forward Neural Network model.

16. The method of claim 9, wherein at least one classifier of the set of classifiers comprises a Long Short-Term Memory Recurrent Neural Network model.
